(19) United States
(12) Patent Application Publication
(10) Pub. No.: US 2010/0287631 A1
(43) Pub. Date: Nov. 11, 2010

(54) WNT PATHWAY MUTATIONS IN CANCER STEM CELLS

(75) Inventors: Catriona Helen M. Jamieson, La Jolla, CA (US); Ifat Geron, San Diego, CA (US); Annelie Abrahamsson, San Diego, CA (US); Edward Kavalerchik, San Diego, CA (US); Irving L. Weissman, Stanford, CA (US); Jason Gotlib, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Regents of the University of California

(21) Appl. No.: 12/447,884

(22) PCT Filed: Nov. 2, 2007
(86) PCT No.: PCT/US07/83532

§ 371 Date: Jun. 15, 2010

Related U.S. Application Data
(60) Provisional application No. 60/856,499, filed on Nov. 2, 2006.

(51) Int. Cl. C12Q 1/48; C12Q 1/68; G01N 33/573; A01K 67/00

(52) U.S. Cl. 800/10; 435/15; 435/6; 435/7.4

(57) ABSTRACT

Cancer specific splicing events in the Wnt/β-catenin signaling pathway are associated with progression of myelogenous leukemia. Misspliced genes of interest include GSK3β. In some embodiments of the invention, polynucleotides are provided that correspond to misspliced GSK3β transcripts associated with cancer. Such transcripts are characterized by a deletion of exon (8), and particularly in exon (8) and (9). Detection of such transcripts in cells is indicative of the presence of leukemia, and particularly of the presence of leukemia stem cells. In other embodiments, polypeptides are provided that are encoded by misspliced GSK3β transcripts associated with cancer. Such polypeptides are useful as diagnostic markers for cancer, and as a target for screening of therapeutic agents. Animal models comprising a human LSC having a misspliced GSK3b transcript provide a useful model for leukemia, for drug/gene screening in the prevention and treatment of leukemia in humans, etc.

Leukemia is a malignant disease of the blood-forming organs, which involves the distorted proliferation and development of white blood cells in bone marrow and blood. Leukemias are usually classified as myelogenous or lymphocytic, according to the types of cells that are involved. Within these groups, there are chronic and acute conditions, which vary in duration and character. Leukemias tend to have age specificity, for example acute lymphoid leukemia generally occurs in young children, while acute myelogenous leukemia is found principally in young adults.

The ability to isolate purified populations of hematopoietic stem cells and myeloid progenitors based on characteristic cell surface (phenotypic) markers has made it possible to identify genes involved in hematopoietic stem cell self-renewal. Normal hematopoietic stem cells, unlike committed hematopoietic progenitors, have the capacity to divide and make identical progeny without undergoing differentiation i.e. self-renewal. Deregulation of self-renewal pathways, which are normally tightly regulated in hematopoietic stem cells, has recently been recognized as an important step in leukemic progression.

Chronic myelogenous leukemia (CML) is a disease having distinct clinical and pathological features. The cause of CML is a specific chromosomal translocation between human chromosomes 9 and 22, resulting in a product commonly referred to as the Philadelphia chromosome. The gene for the tyrosine kinase c-abl resides on the distal arm of human chromosome 9, while the gene for c-bcr resides on human chromosome 22. The translocation places the promoter distal three exons of ABL, including those elements that encode the tyrosine kinase domain, downstream of either the first or second exon of BCR. This chimeric gene, BCR-ABL, encodes a fusion protein often referred to as p185^{bcr-abl} or p210^{bcr-abl}, depending upon the inclusion of the second exon of BCR. p185^{bcr-abl} causes acute leukemia, typically lymphoblastic; p210^{bcr-abl} usually causes CML which may progress to myeloid or lymphoid blast crisis.

Cancer stem cells have been identified, including a leukemic stem cell (LSC). These cells are responsible for disease progression, and for resistance to chemotherapeutic drugs. LSC have a phenotype similar to that of a hematopoietic progenitor cell, which differs from the normal progenitor cells in that the leukemia stem cell has acquired an activated β-catenin pathway. As a result, the LSC have acquired the proliferative and self-renewal capacity that is normally restricted to hematopoietic stem cells. In CML, the LSC responsible for disease progression are phenotypically similar to a granulocyte/macrophage progenitor cell.

Treatment of leukemias has traditionally relied on chemotherapy using anti-neoplastic agents, radiation therapy, corticosteroid therapy and immunotherapy, which may be performed in combination with transplantation of hematopoietic stem cells. Different therapies are utilized depending upon the type of leukemia being treated.

Recently a new class of antiproliferative agents called signal transduction inhibitors has been introduced, which interferes with the pathways that signal the growth of tumor cells. Gleevec (imatinib mesylate) is targeted to the constitutively active abnormal tyrosine kinase created by the Philadelphia chromosome. Imatinib is also an inhibitor of the receptor tyrosine kinases for platelet-derived growth factor (PDGF) and stem cell factor (SCF), c-kit, and inhibits PDGF- and SCF-mediated cellular events.

However, despite the effectiveness of imatinib in inducing both hematologic and cytogenetic remissions in the majority of chronic phase (CP) CML patients, some patients progress, in part as a result of amplification of BCR-ABL and point mutations in the binding site for imatinib on the abl tyrosine kinase active site of P210. In addition, patients who become resistant to Imatinib and develop accelerated phase (AP) or blast crisis (BC) may demonstrate clonal evolution with trisomy 8 and other chromosomal abnormalities suggesting that activation of other oncogenes may contribute to disease progression. Finally, the role of BCR-ABL amplification and additional oncogene activation in HSCs or more committed progenitors with increased proliferative and self-renewal capacity, as a result of aberrant overexpression of hematopoietic stem cell self-renewal genes such as β-catenin, is of great interest.

In another myeloid leukemia, t(8;21) acute myelogenous leukemia, marrow from patients in complete remission contains apparently normal hematopoietic stem cells that produce AML1-ETO transcripts, and their presence during remission implies that such hematopoietic stem cells are pre-leukemic rather than leukemic cells (these transcripts participate in the development of acute myeloid leukemia; AML1-ETO is formed by the fusion of part of the AML1 gene on chromosome 8 with part of the ETO gene on chromosome 21). Similarly, genomic BCR-ABL persists in the marrow of some CML patients who are in a sustained complete cytogenetic remission, and has been detected at very low levels in the leukocytes of healthy individuals, which suggests that pre-leukemic hematopoietic stem cells or more differentiated progenitor cells need additional mutations for progression to overt leukemia.

Bone marrow HSCs are functionally defined by their unique capacity to self-renew and to differentiate to produce all mature blood cell types. In general, the process of development from pluripotent progenitors to mature cells with specific functions involves the progressive loss of developmental potential to other lineages. A hierarchy has emerged in which each successive developmental stage loses the potential to become a specific cell type or class of cells. This stepwise developmental process has been considered linear in the sense that once a cell has made a developmental choice it cannot revert. The earliest known lymphoid-restricted cell in adult mouse bone marrow is the common lymphocyte progenitor (CLP), and the earliest known myeloid-restricted cell is the common myeloid progenitor (CMP). Importantly, these cell populations possess an extremely high level of lineage fidelity in in vitro and in vivo developmental assays.

A description of these cell subsets may be found in Akashi et al. (2000) Nature 404(6774):193, U.S. Pat. No. 6,465,247; and published application U.S. Ser. No. 09/956,279 (common myeloid progenitor); Kondo et al. (1997) Cell 91(5):661-7, and International application WO99/10478 (common lymphoid progenitor); and is reviewed by Kondo et al. (2003) Annu Rev Immunol. 21:759-806, each of which is herein specifically incorporated by reference. International patent application WO 2005/057172 describes profiling of leukemia stem cell and progenitor cell populations, which is herein specifically incorporated by reference, particularly with respect to teaching the phenotype of such cells.

### SUMMARY OF THE INVENTION

Compositions and methods are provided for the analysis and treatment of cancer, particularly hematologic cancers, including myelogenous leukemias. It is shown herein that cancer specific splicing events in the Wnt/β-catenin signaling pathway are associated with progression of myelogenous leukemia. Misspliced genes of interest include GSK3b. It is also demonstrated that down-regulation of axin transcripts is associated with progression of myelogenous leukemia.

In some embodiments of the invention, polynucleotides are provided that correspond to misspliced GSK36 transcripts associated with cancer. Such transcripts are characterized by a deletion of exon 8, or in exon 8 and 9. Of particular interest is an in-frame splice deletion of the GSK36 kinase domain. Detection of such transcripts in cells is indicative of the presence of leukemia, and particularly of the presence of leukemia stem cells.

In other embodiments, polypeptides are provided that are encoded by misspliced GSK3β transcripts associated with cancer. Such polypeptides are useful as diagnostic markers for cancer, and as a target for screening of therapeutic agents.

In another embodiment, animal models are provided for diseases involving leukemia stem cells (LSC). The models are useful for testing and screening of biologically active agents that affect LSC. In the animal models of the invention, a human LSC comprising a misspliced GSK3b transcript is transplanted into an immunocompromised, xenogeneic, non-human recipient, e.g. a rodent. A GSK3b misspliced polynucleotide sequence may be introduced into a suitable cell population, or may be isolated from a source of leukemia cells. The LSC population may further comprise a bioluminescent label, and in some aspects of the invention, in vivo imaging is performed to monitor engraftment. The recipient animal is engrafted with the cell population at a high frequency. The animals provide a useful model for leukemia, for drug/gene screening in the prevention and treatment of leukemia in humans, etc.

### BRIEF DESCRIPTION OF THE DRAWINGS

FIGS. 1A-1D. Blast Crisis CML Progenitors (CD34⁺CD38⁺Lin⁻) Transplant Leukemia A. Neonatal RAG2^{-/-}γ_{c}^{-/-} mice transplanted intrahepatically with blast crisis CML (CD34⁺CD38⁺Lin⁻; 1-4×10⁵) progenitors showed signs of leukemia including wasting, piloerection and lethargy by 6 weeks post-transplant as exemplified by the mouse on the right compared with a no transplant control on the left. B. Transplantation of lentiviral luciferase transduced blast crisis CML (CD34⁺CD38⁺Lin⁻; 1-4×10⁵) progenitors into newborn RAG2^{-/-}γ_{c}^{-/-} mice resulted in prominent tumor bioluminescence as depicted in the bioluminescent image obtained with an IVIS 200 (Caliper Inc) on week 7 post-transplant. C. Representative FACS analysis gated on the live (PI negative) fraction of a tumor single cell suspension from a mouse transplanted with blast phase CML progenitors demonstrated 87.4% human engraftment consisting of 29% human (CD14 and CD33-expressing) myeloid cells compared with no transplant control marrow. D. Left: Photograph of a tumor, in a 35 mm tissue culture dish, derived from a mouse transplanted with blast crisis CML CD34⁺CD38⁺Lin⁻ (1-4×10⁵) cells. There were more BCR-ABL expressing tumors detected in mice transplanted with blast phase CML CD34⁺CD38⁺Lin⁻ cells than in mice transplanted with BC CML CD34₊CD38₋Lin-cells. Middle: Hematoxylyn-eosin stained tumor tissue revealed prominent infiltration with human myeloid cells as typified by the human granulocyte or band (arrow; 100× magnification) characteristic of a blast crisis CML granulocytic sarcoma. Right: Nested primer RT-PCR for P210 BCR-ABL. 1=P210 BCR-ABL Positive Control, CML patient marrow; 2=Negative control, 293T; 3=CD34⁺CD38⁺Thymus; 4=CD34⁺CD38⁺Spleen; 5=CD34⁺CD38⁺Liver; 6=34₊38₊Bone Marrow; 7=CD34⁺CD38⁺Tumor #1; 8=CD34⁺CD38⁺Tumor #2; 9=CD34⁺CD38⁺Tumor #3; 10=CD34⁺CD38⁺Tumor #4; 11=CD34⁺CD38⁺Tumor #5; 12=CD34⁺CD38⁺Tumor #6; 13=Blank; 14=Blank. Of note, there was only 1 BCR-ABL-positive tumor derived from (n=3) mice (n=3) transplanted with CML CD34⁺CD38⁻ cells and 9 BCR-ABL-positive tumors from (n=4) mice transplanted with an equivalent number of CML CD34⁺CD38⁺ cells.

FIGS. 2A-2F. Leukemia Stem Cells are Enriched in the Blast Crisis CML GMP Population. A. In 8 experiments involving normal marrow or cord blood (n=30 mice) and 12 blast crisis CML experiments (n=43 mice) equivalent numbers (10₃-4×10⁵) of HSC, progenitor and blast (Link) cells per experiment were transduced with a lentiviral luciferase-GFP vector. Engraftment kinetics were monitored by weekly bioluminescent imaging. Representative bioluminescent imaging (Xenogen IVIS 200) of neonatal RAG2^{-/-}γ_{c}^{-/-} mice transplanted intrahepatically with 5×10⁴ FACS-purified lentiviral luciferase-GFP transduced CML BC HSC, GMP and lineage-positive (blast) cells demonstrated that blast crisis CML GMP had the greatest engraftment potential. B. In 3 experiments, quantitative bioluminescent engraftment analysis (photons/s/cm²/sr) of equivalent numbers of lentiviral luciferase-GFP transduced blast crisis CML HSC, GMP and Lin⁺ (5×10⁴-10⁵ cells/mouse) demonstrated that GMP had a significantly higher level of bioluminescence (p=0.02; asterisk in figure; two-tailed Student's T-test) than HSC (p=0.06) or Lin⁺ (p=0.35) cells compared with no transplant controls by week 6 following intrahepatic transplantation into newborn RAG2^{-/-}γ_{c}^{-/-} mice. C. Long-term engraftment potential of 2° CML BC GMP and 2° normal HSC was analyzed in 4 experiments. Equivalent numbers of human CD45⁺ cells (10³-10⁴/mouse) were FACS purified from livers, bone marrow and spleens of RAG2^{-/-}γ_{c}^{-/-} mice transplanted with luciferase-GFP transduced normal HSC or CML BC GMP. FACS analysis of primary engraftment revealed comparable human CD45⁺CD38⁺ engraftment between normal HSC and BC CML GMP. D. Bioluminescent imaging was performed at 3 weeks post-transplant and demonstrated that primary normal cord blood CD34⁺ cells (10⁵), 2° normal HSC (10³-10⁴) and 2° CML BC GMP (10³-10⁴; n=6 mice) had short-term engraftment capacity. E. Bioluminescent imaging was performed at 9 weeks post-transplant and demonstrated that both 2° normal HSC and 2° CML BC GMP (n=6 mice) had long-term engraftment capacity. In contrast, 1° normal GMP did not have long-term engraftment potential. No transplant control (upper) and primary normal cord blood CD34⁺ cells served as negative and positive controls for engraftment, respectively. F. Nested primer P210 BCR-ABL RT-PCR analysis of livers from mice transplanted with 2° myeloid BC crisis GMP demonstrated P210 BCR-ABL transcripts (lane 5). There were no detectable BCR-ABL transcripts in livers from mice that were not transplanted (lane 1) or those that were transplanted with normal GMP (lane 2), normal HSC (lane 3), 2° normal HSC (lane 4) or lymphoid blast crisis GMP from P190 BCR-ABL expressing marrow (lane 6).

FIGS. 3A-3E. Aberrant GSK3β Expression by Blast Crisis CML Progenitors. A. Representative FACS plot demonstrating that progression of blast crisis (BC) CML is associated with an expansion of the GMP compartment compared with normal blood and marrow samples and a relative decrease in megakaryocyte-erythroid progenitors (MEP) and common myeloid progenitors (CMP). FACS analysis performed on normal (n=9) and CML chronic phase (CML CP; n=5), CML accelerated phase (CML AP; n=6) and bast crisis CML (BC CML; n=6) blood and marrow cells that were stained with lineage specific antibodies as well as hematopoietic stem cell (HSC) or progenitor specific antibodies revealed that while the proportion of HSC did not expand with progression to BC there was a significant increase in GMP compared with normal controls (p=1.93×10⁶; two-tailed Student's T-test). B. HSC and progenitors were FACS purified from normal or BC CML CD34⁺ (n=3) blood samples and GSK3β transcript levels measured with the aid of quantitative RTPCR. There was a significant difference (p<0.05; two-tailed Student's T-test) in GSK3β transcript levels between normal (mean 0.98+/−S.E. 0.05) and BC CML progenitors (0.36+/−S.E. 0.04). C. FACS histograms of CML chronic phase (green; n=3), accelerated phase (n=2) and blast crisis (n=3) progenitors stained with a rabbit monoclonal antibody specific for the C-terminus of GSK3β that had no cross-reactivity with GSK3β revealed a decrease in GSk3β protein expression with progression to blast crisis. D. Two photon confocal fluorescence microscopic analysis revealed that normal granulocyte-macrophage progenitors (GMP) had little activated β-catenin (Alexa 594-conjugated clone 8E7; red). Conversely, BC CML GMP, with the GSK3β misspliced transcript (see below), had high levels of nuclear (hoechst; blue) activated β-catenin. The plasma membrane is labeled with Alexa-488 conjugated anti-human CD45 green. E. Left: Blast crisis CML HSC in 4 of 7 separate patient samples subjected to DNA sequencing analysis had demonstrable misspliced exon 8 and 9 deleted GSK3β transcripts. Nucleotide sequence data representing two species of GSK3β transcript; the first is the wild type which contains intact exons 8 and 9 and the second species is the mutant form which does not contain exons 8 and 9. The sequencing shown is a reverse primer situated in exon 10 and reading to the 5′ end. The clean sequence on the right is exon 10 and the peak-under-peak is where the deletion begins. Analysis of the peak-under-peak sequence is exon 7 revealing a deletion of exons 8 and 9. Middle: BC CML progenitors in 4 of 7 samples had a deletion of GSK3β exons 8 and 9 in the ORF of the cDNA. Double stranded sequencing of this region reveals that exons 8 and 9 are abolished in the cDNA. The primer located at the 5′ of exon 7 reading to the 3′ direction shows clean exon 7 sequence directly followed by exon 10 sequence and the primer located at the 3′ end of exon 10 reading to the 5′ direction shows clean exon 10 sequence directly followed by exon 7 sequence indicating a complete deletion of exons 8 and 9. Right: BC CML Lineage-positive (blast) cells showing a deletion of GSK3β exon 9 in the ORF of the cDNA that was also detectable in normal samples. Double stranded sequencing of this region reveals that exon 9 is abolished in the cDNA. The primer located at the 5′ end of exon 8 reading to the 3′ direction shows clean exon 8 sequence directly followed by exon 10 sequence and the primer located at the 3′ end of exon 10 reading to the 5′ direction shows clean exon 10 sequence directly followed by exon 8 sequence indicating a complete deletion of exons 8 and 9.

FIGS. 4A-4D. Enhanced Engraftment of Misspliced GSK3β-expressing CML Progenitors A. Left: Nested primer RT-PCR analysis of P210 BCR-ABL transcript levels in hematopoietic tissues including thymus (lane 4; 2° CML 34⁺38⁺ Thymus), spleen (lane 5; 2° CML 34⁺38⁺ Spleen), liver (lane 6; 2° CML 34⁺38⁺ Liver) and bone marrow (lane 7; 2° CML 34⁺38⁺ BM) of RAG2^{-/-}γ_{c}^{-/-} mice transplanted with human CD45⁺ cells from CML 34⁺38⁺ mice (10⁵ cells; n=7 mice). CML patient marrow and 293T cDNA served as positive (lane 2) and negative (lane 3) controls, respectively. Lane 1 contains a 1 Kb DNA ladder. Right: BCR-ABL PCR was positive in 5 of tumors (lanes 2-6) from 2° mice transplanted with CD45⁺ cells derived from 1° mice transplanted with 34⁺38⁺ blast crisis CML progenitors. Water (lane 7; blank) served as a negative control. Lanes 1 and 8 are 100 by DNA ladders. B. FACS analysis of 2° myeloid engraftment in liver and tumor of RAG2^{-/-}γ_{c}^{-/-} mice transplanted with human CD45⁺ cells sorted from bone marrow, liver and spleen of mice transplanted with CML CD34⁺CD38⁺Lin⁻ cells (n=3 experiments). C. FACS analysis revealed enhanced activation of β-catenin in chronic phase CML (n=7) myeloid progenitors lentivirally transduced with misspliced GSK3β-GFP (blue histogram) compared with full-length GSK3β (green histogram), lentiviral GFP alone (red histogram) or cells that were stained with all antibodies except β-catenin (no β-catenin; orange). Results are expressed as percentage of maximum fluorescence intensity. D. Left: Bioluminescent imaging at 3 weeks was compared with 6 weeks (lower panel: n=2 experiments) post-intrahepatic transplantation of RAG2^{-/-}γ_{c}^{-/-} mice (n=8) with chronic phase CML progenitors transduced with lentiviral luciferase alone (CML) or together with misspliced GSK3β (CML+mGSK). Right: Graph of bioluminescent engraftment (photons/sec/cm₂/sr) by chronic phase CML (CP CML; blue line) progenitors (CD34₊CD38₊Lin₋) transduced with lentiviral luciferase GFP alone compared to CP CML lentivirally transduced with both luciferase and misspliced GSK3β-GFP (CP CML+mGSK; red line). Bioluminescent imaging at 3, 5 and 6 weeks post-intrahepatic transplantation of neonatal RAG2^{-/-}γ_{c}^{-/-} mice (n=8). Misspliced GSK3β transduced chronic phase CML progenitors gave rise to higher bioluminescent levels of engraftment at week 6 than luciferase-only transduced controls (*p=0.03; two-tailed Student's T test).

FIGS. 5A-5C. Enhanced Engraftment of β-catenin Expressing Blast Crisis CML CD34⁺CD38⁺Cells A. In Vivo Bioluminescent Human Blast Crisis (BC) CML Progenitor Engraftment Analysis Intrahepatic transplantation of neonatal RAG2^{-/-}γ_{c}^{-/-} mice (n=3 experiments) with no cells, blast crisis (BC) CML CD34⁺CD38⁺Lin⁻ cells (1-4×10⁵) or BC CML CD34⁺CD38⁺Lin⁻ cells (1-4×10⁵) following 48 hour lentiviral luciferase-GFP transduction resulted in engraftment that was detectable by in vivo bioluminescent imaging (IVIS 200, Caliper Inc). Weekly bioluminescent imaging of transplant recipients demonstrated that hepatic engraftment by BC CML CD34⁺CD38⁺Lin⁻-cells (red box) was higher than in CD34⁺CD38⁻Lin⁻ mice (blue box) indicative of dominance of the CD34⁺CD38⁺Lin⁻ fraction within BC CML blood. B. FACS Analysis of Human CML Myeloid Progenitor Hepatic Engraftment Upper: Intrahepatic transplantation of neonatal RAG2^{-/-}γ_{c}^{-/} mice with BC CML CD34₊CD38₋Lin₋ cells 1-4×10⁵) gave rise to myeloid progenitors including common myeloid progenitors (CMP), granulocyte-macrophage progenitors (GMP) and megakaryocyte-erythroid progenitors (MEP) in vivo as determined for human myeloid progenitor antibody stained single cell suspensions of livers with the aid of a FACS Aria. Human antibodies used to detect myeloid progenitors within the propidium idodide, Ter119 negative fraction included CD34-APC, biotinylated CD38 detected with Streptavidin-Cy7-PE, CD123 PE, CD45RA FITC and a Cy5.5PE conjugated lineage cocktail. Negative controls included equivalent number of cells from livers of untransplanted mice and normal human blood served as a positive control. Lower: Intrahepatic transplantation of neonatal RAG2^{-/-}γ_{c}^{-/} mice with BC CML CD34⁺CD38⁺Lin⁻ cells (1-4×10⁵) gave rise to myeloid progenitors in vivo as determined for human myeloid progenitor antibody stained single cell suspensions of livers with the aid of a FACS Aria. C. FACS Analysis of Activated β-catenin Expression by Engrafted Human CML Myeloid Progenitors Upper: Following cell surface staining with antibodies specific for human myeloid progenitor markers (CD34, CD38, CD45RA and CD123), single cell suspensions of livers from mice transplanted with BC CML CD34⁺CD38⁺Lin⁻ cells were fixed with 0.8% paraformaldehyde, permeabilized with 0.3% saponin and stained with an alexa 405 conjugated (Xenon Alexa 405 Mouse IgG labelling kit; Invitrogen) antibody specific for activated β-catenin conjugated to Alexa 405 (clone 8E7, Upstate Inc. Then FACS Aria analysis was performed to detect β-catenin expression by engrafted myeloid progenitors. Lower: The same methodology was used for mice transplanted with BC CML CD34⁺CD38⁺ cells. These experiments demonstrated a 2-fold increase in engrafted myeloid progenitors (standard deviation=0.09) as well as a 1.7 fold (standard deviation=0.07) elevation in activated β-catenin in progenitors derived from transplanted BC CML CD34⁺CD38⁻ cells compared with blast crisis CD34⁺CD38⁻ cells.

FIG. 6. FACS analysis of Blast Crisis (BC) CML Progenitor (CD34⁺CD38⁺Lin⁻) Engraftment Representative FACS analysis (n=3 experiments) of human engraftment in hematopoietic organs including bone marrow, thymus and liver of RAG2^{-/-}γ_{c}^{-/-} transplanted intrahepatically at birth with blast crisis CML CD34⁺CD38⁺Lin⁻-(4×10⁴-4×10⁵) cells showing predominant myeloid (CD14 and CD33) engraftment all hematopoietic organs analyzed (red box). Normal human blood CD45, CD14 and CD33 expression (blue box).

FIGS. 7A-7C. Blast Crisis CML GMP Long-term Engraftment is Comparable to Normal HSC Engraftment. A. In 7 experiments, 26 neonatal RAG2^{-/-}γ_{c}^{-/-} mice were transplanted with untransduced normal cord blood or marrow derived CD34⁺Lin⁻, CD34⁺CD38⁻Lin⁻, CD34⁺CD38⁺Lin⁻, hematopoietic stem cells (HSC), common myeloid progenitors (CMP), granulocyte macrophage progenitors (GMP) or megakaryocyte-erythroid progenitors (MEP). Human engraftment (>1% human CD45 expression) was analyzed between 8 and 12 weeks post transplant by FACS in liver, bone marrow, thymus and spleen. Normal HSC gave rise to the highest levels of engraftment (red; engrafted) while committed myeloid progenitors did not engraft long-term (blue; not engrafted). B. In 8 experiments, 30 neonatal RAG2^{-/-}γ_{c}^{-/} mice were transplanted with normal human hematopoietic stem cells (HSC) or progenitor cells transduced with lentiviral luciferase GFP. Engraftment kinetics were monitored by weekly bioluminescent imaging (IVIS, Caliper Inc). These images demonstrate levels of bioluminescence produced 9 weeks post-intrahepatic transplantation of neonatal immunocompromised (RAG2^{-/-}γ_{c}^{-/-}) mice with HSC derived from three separate normal marrow samples (HSC1, HSC2, HSC3). C. In 6 experiments, 24 neonatal RAG2^{-/-}γ_{c}^{-/-} mice were transplanted with equivalent numbers of BC CML blood or marrow derived HSC, GMP or blasts. Engraftment was analyzed as described above. BC GMP had the most robust long-term engraftment potential. In 12 experiments, 43 neonatal RAG2^{-/-}γ_{c}^{-/-} mice were transplanted with advanced phase lentiviral luciferase transduced HSC, CMP, GMP, MEP or blasts and engraftment kinetics monitored by weekly bioluminescent imaging. Images demonstrate relative levels of bioluminescence at 9 weeks post-transplant for HSC, GMP and blasts.

FIG. 8. GSK3β Single Nucleotide Polymorphism Analysis. A single nucleotide polymorphism (SNP) analysis was conducted on DNA extracted from FACS-purified blast crisis (BC) CML GMP (#1), BC CML Lin₊ (blast) cells (#2) from the same patient, normal lineage positive buffy coat cells (#3), normal cord blood HSC (#4) or K562 cells. Three SNPs, previously shown to induce alternative splicing or enhance transcription of GSK3β in neuronal cells in Parkinson's disease, were identified in BC CML GMP. Blasts that were FACS-purified from BC CML blood harbored two SNPs (RS6438552, RS7633279). Normal HSC had two SNPs (RS334558, RS6438552) while normal Lin⁺ cells lacked all of these GSK3β SNPs. In the initial genomic DNA sequencing screen, a single nucleotide polymorphism (SNP) was detected in APC in normal marrow progenitors and a SNP in c-myc in one blast crisis CML sample (rs4645959).

FIG. 9. Normal Peripheral Blood Progenitors have intact GSK3β Exon 8 HSC, GMP, CD34-enriched and Lin⁺ cells from normal marrow (n=2), cord blood (n=2) and peripheral blood (n=5) samples were subjected to GSK3β cDNA sequencing. This analysis showed a deletion of GSK3β exon 9. Double stranded sequencing of this region revealed that exon 9 is abolished in 20% of transcripts and represents an alternative splice isoform of GSK3β.

### DETAILED DESCRIPTION OF THE EMBODIMENTS

The Wnt/β-catenin signaling pathway has been implicated in the pathogenesis of a variety of tumors. The pathway can be activated as a result of a mutation in the N-terminal Ser 45 position of β-catenin that renders β-catenin impervious to phosphorylation by GSK3b, thus preventing its degradation. Alternatively the pathway can be activated by mutations in APC or axin, two other critical components of the destruction complex.

Herein is described the first targeted cancer stem cell mutation analysis that demonstrates a missplice event. The missplicing results in the production of aberrant GSK3b transcripts, which have, for example, deleted exons 8 and 9. As a result of the missplicing event, there is a decrease in functional GSK3b protein expression. This is the first demonstration of a GSK3b mutation in cancer, and also underscores the importance of epigenetic dysregulation in the pathogenesis of cancer. Detection of GSK3b and other Wnt signaling pathway mediators provides for diagnostic, prognostic and therapeutic methods.

In some embodiments of the invention, polynucleotides are provided that correspond to misspliced GSK3β transcripts associated with cancer. Such transcripts are characterized by a deletion of exon 8, or in exon 8 and 9. Of particular interest is an in-frame splice deletion of the GSK3β kinase domain. Detection of such transcripts in cells is indicative of the presence of leukemia, and particularly of the presence of leukemia stem cells.

In other embodiments, polypeptides are provided that are encoded by misspliced GSK3β transcripts associated with cancer. Such polypeptides are useful as diagnostic markers for cancer, and as a target for screening of therapeutic agents. In other embodiments, over-expression of GSK3β is used to take blast crisis to a less aggressive form. A vector can be used to provide GSK3β over expression, and an in vitro replating assay may be used for detection of the effect, with luciferase expression used for evaluation.

Evidence supports the existence of a rare population of self-renewing cancer stem cells (CSC) that are responsible for cancer progression and therapeutic resistance. Chronic myeloid leukemia (CML) provides a working example for the genetic and epigenetic events involved in CSC production. CML progresses from a chronic phase in hematopoietic stem cells (HSC) that harbor the BCR-ABL translocation, to blast crisis, characterized by aberrant activation of β-catenin within granulocyte-macrophage progenitors (GMP). It is shown herein that blast crisis CML myeloid progenitors can serially transplant leukemia in immunocompromised mice, and thus are enriched for leukemia stem cells (LSC). Genomic DNA and cDNA sequencing analysis revealed that in a majority of blast crisis samples an in-frame splice deletion of the GSK3β kinase domain occurred in blast crisis CML GMP, but not in normal progenitors. CML progenitors with misspliced GSK3β have enhanced β-catenin expression as well as serial engraftment potential.

SNP analysis has revealed SNPs in the GSK3β gene associated with the hematopoietic developmental stage specific missplicing of GSK3β. SNPs of interest include RS334558 (T>C); RS6438552 (T>C); and RS7633297 (A>T). In some embodiments of the invention, a human DNA sample is analyzed for the presence of an SNP associated with GSK3b missplicing, the presence of which is indicative of a predisposition to cancer progression.

Transplantation of blast crisis progenitors expressing misspliced GSK3β produce high levels of myeloid cell engraftment in hematopoietic tissues and tumors derived from transplanted primary and secondary recipient mice. Genetic alteration of chronic phase CML progenitors with misspliced GSK3β sequences led to increased levels of activated β-catenin expression. Chronic phase CML progenitors co-transduced with a detectable marker and misspliced GSK3β had enhanced bioluminescent engraftment compared with control vector transduced progenitors.

In some embodiments, animal models are provided for diseases involving leukemia stem cells (LSC). The models are useful for testing and screening of biologically active agents that affect LSC. In the animal models of the invention, a human LSC comprising a misspliced GSK3b transcript is transplanted into an immunocompromised, xenogeneic, non-human recipient, e.g. a rodent. A GSK3b misspliced polynucleotide sequence may be introduced into a suitable cell population, or may be isolated from a source of leukemia cells. The LSC population may further comprise a bioluminescent label, and in some aspects of the invention, in vivo imaging is performed to monitor engraftment. The recipient animal is engrafted with the cell population at a high frequency. The animals provide a useful model for leukemia, for drug/gene screening in the prevention and treatment of leukemia in humans, etc.

### Wnt/β-Catenin Pathway Genes of Interest

GSK3/3. Glycogen synthase kinase-3 (GSK3) is a proline-directed serine-threonine kinase that was initially identified as a phosphorylating and inactivating glycogen synthase. Two isoforms, alpha and beta, show a high degree of amino acid homology. GSK3B is involved in energy metabolism, neuronal cell development, and body pattern formation. The genetic sequence encoding the 420-amino acid GSK3B protein (GenBank Accession number L33801) is described by Stambolic and Woodgett (1994) Biochem. J. 303: 701-704, herein specifically incorporated by reference. The crystal structure of the protein shows a catalytically active conformation in the absence of activation segment phosphorylation, with the sulfonate of a buffer molecule bridging the activation segment and N-terminal domain in the same way as the phosphate group of the activation segment phospho-ser/thr in other kinases. The location of this oxyanion-binding site in the substrate-binding cleft indicated direct coupling of P+4 phosphate-primed substrate binding and catalytic activation, explained the ability of GSK3B to processively hyperphosphorylate substrates with ser/thr pentad repeats, and suggested a mechanism for autoinhibition in which the phosphorylated N terminus binds as a competitive pseudosubstrate with phospho-ser9 occupying the P+4 site.

Functional SNPs of GSK3B gene include a promoter polymorphism, −50T-C (rs334558), in which the T allele has greater transcriptional activity in vitro, and an intronic polymorphism, −157T-C in intron 5 (rs6438552), in which the T allele is associated with increased levels of GSK3B lacking exons 9 and 11 (see Kwok et al. (2005) Ann. Neurol. 58: 829-839, 2005, herein specifically incorporated by reference.)

The present invention includes a misspliced form of GSK3b which lacks exons 8 and 9. Detection of such a transcript or encoded product is useful in staging leukemias with respect to progression, where the presence of the misspliced transcript or encoded product is indicative of a tendency for the leukemia to progress to blast crisis; and is also indicative of the presence of leukemia stem cells.

With reference to SEQ ID NO:1 (human GSK36 coding sequence), and SEQ ID NO:2 (human GSK36 full length protein), nucleotides 233-1534 are coding sequence. The exon structure is as follows:

exon 1 SEQ ID NO:1, nt. 1-320
exon 2 SEQ ID NO:1, nt. 321-514
exon 3 SEQ ID NO:1, nt. 515-598
exon 4 SEQ ID NO:1, nt. 599-709
exon 5 SEQ ID NO:1, nt. 710-840
exon 6 SEQ ID NO:1, nt. 841-947
exon 7 SEQ ID NO:1, nt. 948-1045
exon 8 SEQ ID NO:1, nt. 1046-1141
exon 9 SEQ ID NO:1, nt. 1142-1180
exon 10 SEQ ID NO:1, nt. 1181-1367
exon 11 SEQ ID NO:1, nt. 1368-1466
exon 12 SEQ ID NO:1, nt. 1467-1626

A variety of methods are useful in detecting the presence of the transcript, including determining the presence of the ratio between a sequence in GSK36 exons not affected by the splice variant, e.g. exons 1, 2, 3, 4, 5, 6, 7, 10, 11, 12 etc., and deleted exons, particularly deleted exon 8. Alternatively detection may be accomplished by detecting the presence of a junction between exon 7 and exon 10, where SEQ ID NO:1 nt. 1045 is adjacent to SEQ ID NO:1, nt. 1181, for example by binding to a probe that spans the junction and that does not bind under the hybridization conditions to the full length transcript sequence. Alternatively detection may be accomplished by determining the distance between a primer that hybridizes to a sequence in any one of the exons 1-7; and any one of exons 10-12, where a decreased distance relative to the full length transcript is indicative of a misplaced variant. For example, PCR amplification may be used to generate an amplification product that spans the region of exons 8 and/or 9, where the size of the amplification product is indicative of whether the sequence includes exons 8 and 9.

In some embodiments of the invention, a polynucleotide is provided that encodes GSK3β Δ exons 8-9, which polynucleotide may be a DNA, RNA, etc., and which may be provided in a vector, including a lentiviral or retroviral vector.

Axin 2. Axin 2 plays an important role in the regulation of the stability of beta-catenin in the Wnt signaling pathway. It organizes a multiprotein complex of APC, beta-catenin, glycogen synthase kinase-3-beta, and axin, which leads to the degradation of beta-catenin. Human cDNA encoding AXIN2 is the human homolog of mouse conductin/rat axil. The 843-amino acid AXIN2 protein contains RGS-signaling, GSK-binding, beta-catenin-binding, and Dsh domains. The AXIN2 gene contains 10 exons and spans more than 25 kb of genomic DNA (see Dong et al. (2001) Cytogenet. Cell Genet. 93: 26-28, herein specifically incorporated by reference).

### Leukemia Stem Cells and Other Hematopoietic Progenitors and Stem Cells

Leukemia stem cells may be responsible for progression and drug resistance in leukemias. LSC have the phenotype similar to that of a hematopoietic progenitor cell, but altered in that the cells have acquired the proliferative and self-renewal capacity that is normally restricted to hematopoietic stem cells.

With respect to myelogenous leukemias, e.g. CML, AML, etc., the phenotype of myeloid lineage progenitors is useful in identification of LSC. These progenitor cells stain negatively for the markers Thy-1 (CD90), IL-7Rα (CD127); and with a panel of lineage markers, which lineage markers may include CD2; CD3; CD4; CD7; CD8; CD10; CD11b; CD14; CD19; CD20; CD56; and glycophorin A (GPA) in humans and CD2; CD3; CD4; CD8; CD19; IgM; Ter110; Gr-1 in mice. With the exception of the mouse MEP subset, all of the progenitor cells are CD34 positive. In the mouse all of the progenitor subsets may be further characterized as Sca-1 negative, (Ly-6E and Ly-6A), and c-kit high. In the human, all three of the subsets are CD38⁺.

In the presence of steel factor (SLF), flt-3 ligand (FL), interleukin (IL)-3, IL-11, GM-CSF, thrombopoietin (Tpo) and erythropoietin (Epo), the CMP cells give rise to various types of myeloerythroid colonies, including CFU-GEMMeg, burst-forming unit-erythroid (BFU-E), CFU-megakaryocytes (CFU-Meg), CFU-granulocyte/macrophage (CFU-GM), CFU-granulocyte (CFU-G) and CFU-macrophage (CFU-M). The GMP subset generates CFU-M, CFU-G, or CFU-GM colonies containing macrophages and/or granulocytes in response to the above growth factors. In contrast, the MEP subset gives rise to CFU-Meg, BFU-E, or CFU-MEP colonies containing only megakaryocytes and/or erythrocytes in response to IL-3, GM-CSF, Tpo and Epo, but do not form colonies in the absence of Tpo and Epo. All three myeloid progenitor populations do not require “early-acting cytokines” such as SLF, FL and IL-11 to initiate colony formation.

All of these progenitors are capable of rapid differentiation activity in vivo. CMP cells give rise to Gr-1+/Mac-1+ myelomonocytic cells and megakaryocytic colonies, as well as TER119+ erythroid cells in spleen and bone marrow. The GMP progenitor population gives rise to Gr-1+/Mac-1+ cells; and the MEP progenitor population to megakaryocytes and erythroid cells.

The LSC identified in CML (CML-LSC) are shown to have a phenotype similar to that of the GMP, in that they are CD34⁺CD38⁺IL-3Rα+CD45RA+ and negative for the panel of lineage markers, which may comprise CD2; CD3; CD4; CD7; CD8; CD10; CD11b; CD14; CD19; CD20; CD56; and glycophorin A (GPA). The cells are capable of self-renewal in vitro; and have an activated β-catenin pathway, which can be inhibited with axin.

Other progenitor subsets that may find use in characterization of LSC include the common lymphoid progenitor, e.g. in analysis of lymphocytic leukemias. Common lymphoid progenitors, CLP, express low levels of c-kit (CD117) on their cell surface. Antibodies that specifically bind c-kit in humans, mice, rats, etc. are known in the art. Alternatively, the c-kit ligand, steel factor (Slf) may be used to identify cells expressing c-kit. The CLP cells express high levels of the IL-7 receptor alpha chain (CDw127). Antibodies that bind to human or to mouse CDw127 are known in the art. Alternatively, the cells are identified by binding of the ligand to the receptor, IL-7.

Human CLPs express low levels of CD34. Antibodies specific for human CD34 are commercially available and well known in the art. See, for example, Chen et al. (1997) Immunol Rev 157:41-51. Human CLP cells are also characterized as CD38 positive and CD10 positive.

The CLP subset also has the phenotype of lacking expression of lineage specific markers, exemplified by B220, CD4, CD8, CD3, Gr-1 and Mac-1. The CLP cells are characterized as lacking expression of Thy-1 (CD90), a marker that is characteristic of hematopoietic stem cells. The phenotype of the CLP may be further characterized as MeI-14⁻, CD43^{lo}, HSA^{lo}, CD45⁺ and common cytokine receptor γ chain positive.

The analysis of megakaryocyte progenitors may also be of interest, for example with respect to megakaryocytic forms of AML. The MKP cells are positive for CD34 expression, and tetraspanin CD9 antigen. The CD9 antigen is a 227-amino acid molecule with 4 hydrophobic domains and 1 N-glycosylation site. The antigen is widely expressed, but is not present on certain progenitor cells in the hematopoietic lineages. The MKP cells express CD41, also referred to as the glycoprotein IIb/IIIa integrin, which is the platelet receptor for fibrinogen and several other extracellular matrix molecules, for which antibodies are commercially available, for example from BD Biosciences, Pharmingen, San Diego, Calif., catalog number 340929, 555466. The MKP cells are positive for expression of CD117, which recognizes the receptor tyrosine kinase c-Kit. Antibodies are commercially available, for example from BD Biosciences, Pharmingen, San Diego, Calif., Cat. No. 340529. MKP cells are also lineage negative, and negative for expression of Thy-1 (CD90).

### Leukemias

Chronic leukemias include chronic myelogenous leukemia (CML); chronic myelomonocytic leukemia, and chronic lymphocytic leukemia. Clonal myeloproliferation of CML is caused by malignant transformation of an early hematopoietic cell, and is characterized clinically by striking overproduction of granulocytes, primarily in the bone marrow but also in extramedullary sites. The neoplastic clone may include RBC, megakaryocyte, monocyte, and even some T and B cells. Normal stem cells are retained and can emerge after drug suppression of the CML clone. In most patients, the CML clone progresses to an accelerated phase and final blast crisis.

In the symptomatic patient, the WBC count is usually elevated and left shifted. The platelet count is normal or moderately increased. On blood smears, all stages of granulocyte differentiation are seen. The absolute eosinophil and basophil concentrations can be strikingly increased, but the absolute lymphocyte and monocyte concentrations may be normal. The bone marrow is hypercellular on aspirate and biopsy. The Philadelphia chromosome can be demonstrated in almost all patients by chromosomal analysis.

During the accelerated phase of disease progression, anemia and thrombocytopenia develop, basophils may increase, granulocyte maturation may be defective and the proportion of immature cells may increase. Further evolution may lead to a blast crisis with myeloblasts, lymphoblasts, or megakaryoblasts.

Imatinib mesylate is the drug of choice for most cases, although patients may also be treated with interferon, hydroxyurea, cytarabine, and other myelosuppressive drugs such as 6-mercaptopurine, 6-thioguanine, melphalan, and cyclophosphamide. In the absence of hematopoietic progenitor cell transplantation, for most cases the Ph-positive clone persists in the marrow.

Chronic Myelomonocytic Leukemias (CMML) include two types: an adult type referred to as CMML and a form of childhood leukemia called Juvenile Myelomonocytic Leukemia (JMML) or Juvenile Chronic Myelogenous Leukemia (JCML). CMML leukemias have features that are characteristic of myelogenous leukemia. CMML is more rapidly progressive than “typical” chronic myelogenous leukemia and less rapidly progressive than acute myelomonocytic leukemia.

JMML occurs most often in infants and children under four years of age. The blood cell and bone marrow findings are similar in some respects to adult chronic myelomonocytic leukemia. Both disorders are cancers that originate in a marrow cell. Infants with JMML fail to thrive. Low hemoglobin (anemia), low platelets, and moderate increases in white cell count are common. The blood invariably has an increased concentration of monocytes and immature granulocytes (myelocytes), hence the term “myelomonocytic” leukemia. The Ph chromosome, characteristic of typical chronic myelogenous leukemia is not present. JMML has been resistant to chemotherapy. The median survival of patients with the juvenile form of the disease is usually less than two years.

Adult type chronic myelomonocytic leukemia is part of the spectrum of myeloproliferative diseases that may have findings that simulate typical chronic myelogenous leukemia (CML) such as anemia, high white cell count and enlargement of the spleen except that there is an increase in monocytes rather than granulocytes. The cells do not contain the Ph chromosome, or BCR-ABL oncogene, that characterizes chronic myelogenous leukemia. Most patients with chronic myelomonocytic leukemia (CMML) are over 50 years of age. Blood cell counts may be variable with CMML. The white blood cell count may be slightly decreased, normal, or moderately elevated. There are two forms of CMML—a dysplastic form, MDS-CMML, with less than 13×10⁹/L monocytes versus a proliferative form, MPD-CMML, with >13×10⁹/L monocytes. Blood myeloblasts may be absent or, when present, are in low proportions. In some cases, a translocation of chromosomes 5 and 12, occurs, resulting in the PDGFR-β-TEL gene translocation.

Chronic leukocytic leukemia (CLL) is the clonal expansion of mature-appearing lymphocytes involving bone marrow, lymph nodes and other lymphoid tissues with progressive infiltration of the liver and spleen. Most cases are diagnosed in elderly patients. Lymphocyte accumulation probably begins in the bone marrow and spreads to lymph nodes and other lymphoid tissues. Usually in late disease, abnormal hematopoiesis results in anemia, neutropenia, thrombocytopenia, and decreased immunoglobulin production. Traditional delineation of CLL has been of the most common subtype (B-cell form), which represents almost all cases, and a rare T cell type. In addition, other chronic leukemic patterns have been categorized under CLL: prolymphocytic leukemia, leukemic phase of cutaneous T-cell lymphoma, hairy cell leukemia, and lymphoma leukemia.

The hallmark of CLL is sustained, absolute lymphocytosis and increased lymphocytes in the bone marrow. CBC and bone marrow aspiration confirm diagnosis. Although CLL is progressive, some patients may be asymptomatic for years; therapy is not indicated until active progression or symptoms occur. Specific therapy includes chemotherapy, corticosteroids, and radiotherapy. Alkylating drugs, especially chlorambucil, alone or with corticosteroids, fludarabine, cyclophosphamide, pentostatin, rituximab and campath are also of use.

### Methods and Compositions

The GSK3β splice variants and encoded proteins described herein are useful in the identification of individuals predisposed to progression of cancer, particularly in the testing of early stage cancer samples, for example chronic stage leukemias, such a myelogenous leukemias. Cancers of interest include those associated with aberrant wnt signaling. The genetic sequences of the invention are also of interest for the identification of leukemia stem cells, which cells provide a target for drug screening and other therapies directed at cancer stem cells. The genetic sequences may be naturally present or introduced into a leukemia cell, e.g. a CML cell with the phenotype of being CD34+CD38+Lin⁻, which cells may be transplanted into a xenogeneic host as an animal model for leukemia. The encoded proteins are useful as an immunogen to raise specific antibodies, in drug screening for compositions that mimic or modulate activity or expression, including altered forms of the proteins, and as a therapeutic.

In one aspect, the invention provides for an isolated nucleic acid molecule other than a naturally occurring chromosome comprising a sequence encoding a GSK3β Δ exons 8-9 protein, including polymorphic variants, or a homolog or variant thereof, which variant may be associated with cancer progression. The nucleic acid may be operably linked to a vector and/or control sequences for expression in a homologous or heterologous host cell. Such a host cell can find use in the production of the encoded protein.

The DNA sequence encoding GSK3β Δ exons 8-9 polypeptides may be RNA or DNA or a fragment thereof. Fragments of interest for probes, producing polypeptides, etc. will comprise a junction not found in the full-length protein. The term GSK3β gene shall be intended to mean the open reading frame encoding any one of the specific GSK3β polypeptides, introns, as well as adjacent 5′ and 3′ non-coding nucleotide sequences involved in the regulation of expression, up to about 1 kb beyond the coding region, but possibly further in either direction. The gene may be introduced into an appropriate vector for extrachromosomal maintenance or for integration into the host.

The GSK38 sequences are isolated and obtained in substantial purity, generally as other than an intact mammalian chromosome. Usually, the DNA will be obtained substantially free of other nucleic acid sequences that do not include an GSK3B sequence or fragment thereof, generally being at least about 50%, usually at least about 90% pure and are typically recombinant, i.e. flanked by one or more nucleotides with which it is not normally associated on a naturally occurring chromosome.

The subject genetic sequences may be employed for synthesis of a GSK38 splice variant, e.g. GSK38 Δ exons 8-9, or polypeptide fragments thereof, particularly fragments corresponding to functional domains; binding sites; etc.; and including fusions of the subject polypeptides to other proteins or parts thereof. For expression, an expression cassette may be employed, providing for a transcriptional and translational initiation region, which may be inducible or constitutive, where the coding region is operably linked under the transcriptional control of the transcriptional initiation region, and a transcriptional and translational termination region. Various transcriptional initiation regions may be employed that are functional in the expression host. Polypeptides of particular interest that are fragments of the GSK38 splice variants include the junction formed between exon 7 and exon 10.

The polypeptides may be expressed in prokaryotes or eukaryotes in accordance with conventional ways, depending upon the purpose for expression. For large scale production of the protein, a unicellular organism, such as E. coli, B. subtilis, S. cerevisiae, or cells of a higher organism such as vertebrates, particularly mammals, e.g. COS 7 cells, may be used as the expression host cells. In many situations, it may be desirable to express the GSK38 splice variant gene in mammalian cells, where the gene will benefit from native folding and post-translational modifications. Small peptides can also be synthesized in the laboratory, including specific peptide epitopes, domains, and the like, where peptides will usually be at least about 8 amino acids in length, more usually at least about 20 amino acids in length, up to complete domains, and the full length protein. Also included are fusion proteins, where all or a fragment of the GSK3B protein is fused to a heterologous polypeptide, e.g. green fluorescent protein, antibody Fc regions, poly-histidine, and the like.

In mammalian host cells, a number of viral-based expression systems may be used, including retrovirus, lentivirus, adenovirus, adeno-associated virus, and the like. In cases where a lentivirus is used as an expression vector, the coding sequence of interest can be ligated to an lentivirus transcription/translation control complex. This chimeric gene may then be inserted in the lentivirus genome by in vitro or in vivo recombination.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

With the availability of the polypeptides in large amounts, by employing an expression host, the polypeptides may be isolated and purified in accordance with conventional ways. A lysate may be prepared of the expression host and the lysate purified using HPLC, exclusion chromatography, gel electrophoresis, affinity chromatography, or other purification technique. The purified polypeptide will generally be at least about 80% pure, preferably at least about 90% pure, and may be up to and including 100% pure. Pure is intended to mean free of other proteins, as well as cellular debris.

The polypeptide may be labeled, either directly or indirectly. Any of a variety of suitable labeling systems may be used, including but not limited to, radioisotopes such as ¹²⁵I; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library.

### Diagnostic Assays

Screening of leukemia or cancer associated with GSK3β splicing may be performed by protein, DNA or RNA sequence and/or hybridization analysis of any convenient sample from a patient, e.g. biopsy material, blood sample, scrapings from cheek, etc. A nucleic acid sample from a patient is analyzed for the presence of a GSK3β splice variant, e.g. a deletion including exon 8. Individuals are screened by analyzing mRNA for the presence of a splice variant.

A number of methods are available for analyzing nucleic acids for the presence of a specific sequence. Cells that express GSK3β genes may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. (1985) Science 239:487, and a review of current techniques may be found in Sambrook, et al. Molecular Cloning: A Laboratory Manual, CSH Press 1989, pp. 14.2-14.33. Amplification may also be used to determine whether a polymorphism is present, by using a primer that is specific for the polymorphism. Alternatively, various methods are known in the art that utilize oligonucleotide ligation as a means of detecting polymorphisms, for examples see Riley et al. (1990) N.A.R. 18:2887-2890; and Delahunty et al. (1996) Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. The nucleic acid may be sequenced by dideoxy or other methods. Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in WO95/35505, may also be used as a means of detecting the presence of variant sequences. Single strand conformational polymorphism (SSCP) analysis, denaturing gradient gel electrophoresis (DGGE), mismatch cleavage detection, and heteroduplex analysis in gel matrices are used to detect conformational changes created by DNA sequence variation as alterations in electrophoretic mobility. Alternatively, where splicing creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels.

The hybridization pattern of a sequence to an array of oligonucleotide probes immobilized on a solid support, as described in U.S. Pat. No. 5,445,934, or in WO95/35505, may be used as a means of detecting the presence of splice variants. In one embodiment of the invention, an array of oligonucleotides are provided, where discrete positions on the array are complementary to at least a portion of mRNA of GSK3β. Such an array may comprise a series of oligonucleotides, each of which can specifically hybridize to a nucleic acid, e.g. mRNA, cDNA, genomic DNA, etc. from a GSK3β exon.

Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity may be determined by hybridization under stringent conditions, for example, at 50° C. or higher and 0.1×SSC (9 mM saline/0.9 mM sodium citrate).

Screening may also be based on the functional or antigenic characteristics of the protein. Immunoassays designed to detect splicing variation in GSK3β proteins may be used in screening. Antibodies specific for GSK3β may be used in screening immunoassays. The presence of cancer associated splice variation in a cancer cell indicates the presence of leukemia stem cells, and a tendency for progression of the cancer.

Diagnosis may be performed by a number of methods. The different methods all determine the absence or presence or altered amounts of misspliced GSK3β in patient cells. For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and GSK3β splice variant in a lysate. Measuring the concentration of GSK3β binding in a sample or fraction thereof may be accomplished by a variety of specific assays. A conventional sandwich type assay may be used. For example, a sandwich assay may first attach GSK3B-specific antibodies to an insoluble surface or support. The particular manner of binding is not crucial so long as it is compatible with the reagents and overall methods of the invention. They may be bound to the plates covalently or non-covalently, preferably non-covalently.

Other immunoassays are known in the art and may find use as diagnostics. Ouchterlony plates provide a simple determination of antibody binding. Western blots may be performed on protein gels or protein spots on filters, using a detection system specific for GSK3β splicing as desired, conveniently using a labeling method as described for the sandwich assay.

For assessment of the presence of mRNA or protein, a sample is taken from a patient suspected of having cancer. Samples, as used herein, include biological fluids such as tracheal lavage, blood, cerebrospinal fluid, tears, saliva, lymph, dialysis fluid and the like; organ or tissue culture derived fluids; and fluids extracted from physiological tissues. Also included in the term are derivatives and fractions of such fluids. Biopsy samples and blood samples are of particular interest, e.g. a tumor biopsy sample, etc. The number of cells in a sample will generally be at least about 10³, usually at least 10⁴ more usually at least about 10⁵. The cells may be dissociated, in the case of solid tissues, or tissue sections may be analyzed. Alternatively a lysate of the cells may be prepared.

### Compound Screening

One can identify ligands or substrates that bind to, modulate or mimic the action of GSK3β. Of particular interest are compounds that upregulate expression of biologically active, full-length GSK3β. Also of interest are agents that specifically bind to GSK3β Δ exons 8-9, which are useful in targeting leukemia stem cells. Screening may utilize GSK3β Δ exons 8-9 genetic sequences or polypeptide sequences, and may further utilize animal models of the invention comprising human GSK3β Δ exons 8-9 sequences. Agents of interest may include, in addition to small organic molecules; polypeptides and fragments thereof; antibodies and derivatives thereof; and the like.

Areas of investigation are the development of treatments for cancer, particularly leukemia, and more particularly CML and AML. Drug screening identifies agents that provide targeting, inhibition, replacement, or enhancement for GSK3β function in affected cells.

Of particular interest are screening assays for agents that have a low toxicity for human cells. A wide variety of assays may be used for this purpose, including labeled in vitro protein-protein binding assays, protein-DNA binding assays, electrophoretic mobility shift assays, immunoassays for protein binding, cell culture and animal assays, and the like. The purified protein may also be used for determination of three-dimensional crystal structure, which can be used for modeling intermolecular interactions, transcriptional regulation, etc.

The term “agent” as used herein describes any molecule, e.g. protein or pharmaceutical, with the capability of binding to, altering or mimicking the physiological function of GSK3β. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Other assays of interest detect agents that mimic GSK3β function. For example, candidate agents are added to a cell that lacks functional GSK3β, and screened for the ability to reproduce GSK3β in a functional assay.

### Therapeutic Methods

Agents that modulate activity of GSK3β genes or proteins in cancer cells, e.g. in leukemia stem cells, provide a point of therapeutic or prophylactic intervention, particularly agents that upregulate activity of the polypeptide, or expression of the gene. Agents of interest may directly modulate GSK3β gene expression, e.g. expression vectors, etc.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

The term “unit dosage form” as used herein refers to physically discrete units suitable as unitary dosages for subjects (e.g., animals, usually humans), each unit containing a predetermined quantity of agent(s) in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

Methods can be designed to selectively deliver nucleic acids to certain cells. Examples of such cells include LSC, etc. Certain treatment methods are designed to selectively express an expression vector to cells of interest. One technique for achieving selective expression in nerve cells is to operably link the coding sequence to a promoter that is primarily active in LSC, e.g. a β-catenin activated promoter, and the like. Alternatively, or in addition, the nucleic acid can be administered with an agent that targets the nucleic acid to the cells of interest. For instance, the nucleic acid can be administered with an antibody that specifically binds to a cell-surface antigen. When liposomes are utilized, substrates that bind to a cell-surface membrane protein associated with endocytosis can be attached to the liposome to target the liposome to nerve cells and to facilitate uptake.

The polypeptides, nucleic acids, or other compounds having a desired pharmacological activity may be administered in a physiologically acceptable carrier to a host. Such therapeutic agents may be administered in a variety of ways, orally, topically, parenterally e.g. subcutaneously, intraperitoneally, by viral infection, intravascularly, etc. Inhaled treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### Animal Models

Leukemia cells, particularly human cells having a GMP phenotype, e.g. CD34⁺CD38⁺Lin⁻ may express a GSK3β splice variant, or may be genetically altered, e.g. by lentiviral transduction, introduction of plasmids or other vectors, including viral vectors such as AAV, adenovirus, and the like to express a GSK3β splice variant. Genes of interest include, without limitation, human GSK3β Δ exons 8-9, etc. The cells are then transplanted into an immunocompromised non-human host, e.g. (RAG2^{-/-}γ_{c}^{-/-} mice, SCID mice, etc.) The cells may be introduced intrahepatically at birth, or at other suitable times and routes, e.g. i.v., etc. Preferably the cells provide for a detectable marker, particularly a biolumiscent marker, e.g. luciferase, GFP and the like, and may be tracked in vivo via bioluminescent imaging (Caliper IVIS 200), by FACS analysis on hematopoietic organs including liver, spleen, marrow and thymus, and the like.

The stem and/or progenitor cells may be obtained from any mammalian species, e.g. human, equine, bovine, porcine, canine, feline, rodent, e.g. mice, rats, hamster, primate, etc., usually human. The tissue may be obtained by blood draw, biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−180° C.) indefinitely. The population of stem and/or progenitor cells may be enriched for a population of interest, although such an enrichment step is not required.

The cell population can be selectively enriched for a cell of interest, e.g. LSC, HSC, CMP, GMP, MEP, etc., using any combination of markers as described above for selective enrichment, e.g. using magnetic sorting techniques, flow cytometry, etc. as known in the art. In some embodiments the cells are enriched for expression of CD34, e.g. by immunomagnetic selection, flow cytometry, etc. Selection may be performed before or after introduction and expression of a nucleic acid construct.

Separation of the desired cells for engraftment may be performed using affinity separation to provide an enriched cell population with a phenotype of interest. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, LDS). Any technique may be employed which is not unduly detrimental to the viability of the selected cells.

Of particular interest is the use of antibodies as affinity reagents. Conveniently, these antibodies are conjugated with a label for use in separation or used in conjunction with a labeled second antibody that binds to them. Labels include magnetic beads, which allow for direct separation; biotin, which can be bound to avidin or streptavidin bound to a support; fluorochromes, which can be used with a fluorescence activated cell sorter; or the like, to allow for ease of separation of the particular cell type. Fluorochromes that find use include phycobiliproteins, e.g. phycoerythrin and allophycocyanins, fluorescein and Texas red.

A nucleic acid construct is introduced into the above-described cell or population of cells, where the construct comprises sequences of a GSK3β splice variant. The coding sequence is operably linked to a promoter, which may be a constitutive or inducible promoter, and may be the native promoter for the gene of interest, or may be heterologous relative to the coding sequence.

A variety of vectors are known in the art for the delivery of sequences into a cell, including plasmid vectors, viral vectors, and the like. In a preferred embodiment, the vector is a retroviral or lentiviral vector. For example, see Baum et al. (1996) J Hematother 5(4):323-9; Schwarzenberger et al. (1996) Blood 87:472-478; Nolta et al. (1996) P.N.A.S. 93:2414-2419; and Maze et al. (1996) P.N.A.S. 93:206-210, Mochizuki et al. (1998) J Virol 72(11):8873-83. The use of adenovirus based vectors with hematopoietic cells has also been published, see Ogniben and Haas (1998) Recent Results Cancer Res 144:86-92.

Combinations of retroviruses and an appropriate packaging line may be used, where the capsid proteins will be functional for infecting the target cells. Usually, the cells and virus will be incubated for at least about 24 hours in the culture medium. Commonly used retroviral vectors are “defective”, i.e. unable to produce viral proteins required for productive infection. Replication of the vector requires growth in the packaging cell line.

Various techniques known in the art may be used to transfect the target cells, e.g. electroporation, calcium precipitated DNA, fusion, transfection, lipofection and the like. The particular manner in which the DNA is introduced is not critical to the practice of the invention.

The nucleic acid construct may further comprise a detectable marker. Viable cells expressing the marker can also be sorted, in order to isolate or enrich for the cells of interest. Many such markers are known in the art, for example antibiotic resistance, color change of a substrate, expression of a recombinase, e.g. cre recombinase, FLP recombinase, pSR1 recombinase, etc., which is indirectly detected; expression of luminescence producing proteins, e.g. luciferase, green fluorescent proteins, etc.

In a preferred embodiment of the invention, the marker is a luminescence producing protein, preferably green fluorescent protein (GFP) and/or luciferase. Luciferase, e.g. firefly luciferase enzyme, operably linked to a constitutive or inducible promoter allows in vivo bioluminescence detection of transfected cells, for example using a CCCD camera after luciferin administration. See Contag et al. (1998) Nat. Med. 4:245, herein specifically incorporated by reference for teaching the use of luciferase transgenes.

The native gene encoding GFP has been cloned from the bioluminescent jellyfish Aequorea victoria (Morin, J. et al., J Cell Physiol (1972) 77:313-318). The availability of the gene has made it possible to use GFP as a marker for gene expression. GFP itself is a 283 amino acid protein with a molecular weight of 27 kD. It requires no additional proteins from its native source nor does it require substrates or cofactors available only in its native source in order to fluoresce. Mutants of the GFP gene have been found useful to enhance expression and to modify excitation and fluorescence. GFP-S65T (wherein serine at 65 is replaced with threonine) may be used, which has a single excitation peak at 490 nm. (Heim, R. et al., Nature (1995) 373:663-664); U.S. Pat. No. 5,625,048. Other mutants have also been disclosed by Delagrade, S. et al., Biotechnology (1995) 13:151-154; Cormack, B. et al., Gene (1996) 173:33-38 and Cramer, A. et al. Nature Biotechnol (1996) 14:315-319. Additional mutants are also disclosed in U.S. Pat. No. 5,625,048. By suitable modification, the spectrum of light emitted by the GFP can be altered.

The expression of the detectable marker, where the marker is a fluorescent protein, can be monitored by CCCD camera, flow cytometry, where lasers detect the quantitative levels of fluorophore. Flow cytometry, or FACS, can also be used to separate cell populations based on the intensity of fluorescence, as well as other parameters such as cell size and light scatter. Although the absolute level of staining may differ, the data can be normalized to a control.

The cells are introduced into a suitable xenogeneic immunocompromised animal host. Any suitable site for injection may be used, e.g. intravenous, or to a hematopoietic organ, e.g. intrathymic, intra-marrow, intrasplenic, intrahepatic, etc. Various numbers of cells are introduced, depending on the specific use of the animal model, and the purity of cells being transplanted, where highly purified cell populations may be administered in lower numbers than unselected populations. Usually at least about 10, 10², 10³, 10⁴, 10⁵ 10⁶ or more cells are introduced.

Immunocompromised mammalian hosts suitable for implantation and having the desired immune incapacity exist or can be created. The significant factor is that the immunocompromised host is incapable of mounting an immune response against the introduced cells. Of particular interest are small mammals, e.g. rabbits, gerbils, hamsters, guinea pigs, etc., particularly rodents, e.g. mouse and rat, which are immunocompromised due to a genetic defect that results in an inability to undergo germline DNA rearrangement at the loci encoding immunoglobulins and T-cell antigen receptors or to a genetic defect in thymus development (nu/nu). Mazurier (1999) J Interferon Cytokine Res 19: 533-41 developed an immunodeficient mouse model by combining recombinase activating gene-2 (RAG2) and common cytokine receptor gamma chain (gamma c) mutations. The RAG2^{-/-}γ_{c}^{-/-} double mutant mice are completely alymphoid (T-, B-, NK-), show no spontaneous tumor formation, and exhibit normal hematopoietic parameters, and are of particular interest.

Presently available hosts also include mice that have been genetically engineered by transgenic disruption to lack the recombinase function associated with RAG-1 and/or RAG-2 (e.g. commercially available TIM™ RAG-2 transgenic), to lack Class I and/or Class II MHC antigens (e.g. the commercially available C1D and C2D transgenic strains), or to lack expression of the Bcl-2 proto-oncogene. In other embodiments the host animals are mice that have a homozygous mutation at the scid locus, causing a severe combined immunodeficiency which is manifested by a lack of functionally recombined immunoglobulin and T-cell receptor genes. The scid/scid mutation is available or may be bred into a number of different genetic backgrounds, e.g. CB.17, ICR (outbred), C3H, BALB/c, C57B1/6, AKR, BA, B10, 129, etc. Other mice which are useful as recipients are NOD scid/scid; SGB scid/scid, bh/bh; CB.17 scid/hr; NIH-3 bg/nu/xid and META nu/nu. Transgenic mice, rats and pigs are available which lack functional B cells and T cells due to a homozygous disruption in the CD3ε gene. Immunocompromised rats include HsdHan:RNU-rnu; HsdHan:RNU-rnu/+; HsdHan:NZNU-rnu; HsdHan:NZN U-rnu/+; LEW/HanHsd-rnu; LEW/HanHsd-rnu/α; WAG/HanHsd-rnu and WAG/HanHsd-rnu/+.

The host may be neonate, or newborn to young adult, usually less than about 6 weeks of age, and may be less than about 4 weeks of age, less than about 2 weeks of age at implantation. The mammalian host will be grown in conventional ways. Depending on the degree of immunocompromised status of the mammalian host, it may be protected to varying degrees from infection. An aseptic environment is indicated. Prophylactic antibiosis may be used for protection from infection. Alternatively, it may be satisfactory to isolate the potential hosts from other animals in gnotobiotic environments after cesarean derivation. The feeding and maintenance of the host will for the most part follow gnotobiotic techniques.

The subject GSK3β splice variant expressing cells and animal models are useful for in vitro and in vivo assays and screening to detect factors that are active in influencing growth of leukemia stem cells. The cells find use in assessing pre-clinical efficacy of anti-cancer drugs alone and in combination with other agents.

In screening assays of particular interest, an agent is administered to the animal, which may be administered at a dose effective to substantially alter GSK3β splice variant activity, or may be administered at various concentrations. In some embodiments, the animals or a panel of animals are contacted with a candidate agent for the treatment of leukemia, where the candidate agent is administered to the animal in a suitable manner, e.g. orally, by injection, inhalation, etc., and the effect on LSC is examined following such administration, by determining the quantity and/or characteristics of progeny from the GSK3β splice variant expressing xenogeneic cells.

The animal models of the invention may be used to test novel therapeutics directed against GSK3β splice variants. Of particular interest are screening assays for agents that are active on human cells. A wide variety of assays may be used for this purpose, including immunoassays for protein binding; determination of cell growth, differentiation and functional activity, and the like.

Parameters of interest for assessment of a candidate agent include the quantitation of xenogeneic leukemia cells, which may be quantitated by the number of GSK3β splice variant expressing cells. Various hematopoietic organs may be collectively or individually assessed in this manner, e.g. blood, spleen, liver, bone marrow, thymus, lymph nodes, and the like.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the array” includes reference to one or more arrays and equivalents thereof known to those skilled in the art, and so forth. All technical and scientific terms used herein have the same meaning as commonly understood to one of ordinary skill in the art to which this invention belongs unless clearly indicated otherwise.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Glycogen Synthase Kinase 3 eta Missplicing Contributes to Chronic Myeloid Leukemia Stem Cell Generation

Recent evidence suggests that a rare population of self-renewing cancer stem cells (CSC) is responsible for cancer progression and therapeutic resistance. Chronic myeloid leukemia (CML) represents an important paradigm for understanding the genetic and epigenetic events involved in CSC production. CML progresses from a chronic phase in hematopoietic stem cells (HSC) that harbor the BCR-ABL translocation, to blast crisis, characterized by aberrant activation of β-catenin within granulocyte-macrophage progenitors (GMP). Here we show that blast crisis CML myeloid progenitors can serially transplant leukemia in immunocompromised mice, and thus are enriched for leukemia stem cells (LSC). Genomic DNA and cDNA sequencing analysis of Wnt/β-catenin pathway regulatory genes including APC, GSK3β, axin 1, β-catenin, LEF-1, cyclin D1 and c-myc in 49 normal and 51 CML HSC, GMP and lineage-positive populations revealed that in 57% of blast crisis samples a novel in-frame splice deletion of the GSK3β kinase domain occurred in blast crisis CML GMP, but not blasts found in blast crisis or normal progenitors. CML progenitors with misspliced GSK3β have enhanced β-catenin expression as well as serial engraftment potential.

These data demonstrate although that chronic phase CML is initiated by BCR-ABL expression in an HSC clone, progression to blast crisis may include missplicing of GSK3β in GMP LSC, enabling unphosphorylated β-catenin to participate in LSC self-renewal. Missplicing of GSK3β represents a unique mechanism for the emergence of blast crisis CML LSC, is a potential diagnostic indicator of disease progression and represents a novel therapeutic target in blast crisis CML. As the first description of GSK3β dysregulation in cancer, this research provides a focal point for investigating whether GSK3β missplicing also triggers β-catenin activation in other CSC.

Chronic myeloid leukemia (CML) was the first cancer shown to be initiated at the hematopoietic stem cell (HSC) level by a pathognomonic chromosomal abnormality, the Philadelphia chromosome, that produces a fusion gene, BCR-ABL, and its constitutively active protein tyrosine kinase —P²¹⁰BCR-ABL. CML was also the first malignancy treated with a molecularly targeted agent, imatinib, which inhibits the BCR-ABL tyrosine kinase. However, most BCR-ABL inhibitor treated CML patients harbor cells with low-level BCR-ABL transcripts suggesting that these cells may be susceptible to further transforming events spawning future relapse.

Several studies indicate that relapse and disease progression derive from a rare population of cancer stem cells (CSC), the only cells within the cancer that can recapitulate the tumor in transplant models. Recent evidence suggests that CSC are generated by a sequence of heritable events, both epigenetic and mutational, which alter progenitor self-renewal, survival and differentiation. To date, CSC have been identified in human acute myelogenous leukemia (AML), breast cancer, several brain tumors, head and neck squamous cell carcinomas, pancreatic and colon cancer. Preliminary studies suggest that CSC of the hematopoietic system, also called leukemia stem cells (LSC), have subverted the properties normally ascribed to HSC such as self-renewal capacity.

By studying 100 CML blood and marrow samples, we discovered that a population that shared the same immunophenotype as granulocyte macrophage progenitors (GMP), expressed high levels of BCR-ABL and had activated the Wnt/β-catenin self-renewal pathway. These candidate LSC had enhanced replating capacity, an in vitro surrogate measure of self-renewal potential that was potently inhibited by a specific Wnt pathway antagonist-axin.

Here we studied 1) the capacity of candidate blast crisis (BC) LSC to self-renew in immunocompromised mice and 2) the mechanisms driving Wnt/β-catenin self-renewal pathway activation. We developed an in vivo bioluminescent BC LSC model via intrahepatic transplantation of lentiviral luciferase transduced candidate BC LSC into neonatal immunocompromised (RAG2^{-/-}γ_{c}^{-/-}) mice. Controls included CML HSCs (Table 1) and blast (Link) cells as well as normal HSCs and progenitors.

Supplemental TABLE 1
Patient Characteristics

| | | | | WBC count | % PB | Treatment at Time |
|---|---|---|---|---|---|---|
| Patient Number | Sex/Age | Date | Phase of CML | [K/mm³] | Blasts | of Sample |
| 01 | F/45 | May 30, 2006 | Chronic phase | 35.6 | 1 | Nilotinib |
| 02 | F/72 | Mar. 8, 2005 | Chronic phase | 17.8 | 0 | Imatinib 400 mg daily |
| 03 | F/59 | Apr. 6, 2005 | Chronic phase | 88.3 | 1 | Imatinib intolerant; |
| 04 | F/50 | Jun. 28, 2005 | Chronic phase | 44.2 | 1 | Imatinib intolerant |
| | | | | | | stopped 2 months prior |
| 05 | M/52 | Mar. 07, 2006 | Accelerated phase | 3.3 | 2 | MK-0457 (VX-680) |
| 06 | M/59 | Jan. 25, 2001 | Myeloid blast crisis | 14.7 | 87 | Hydroxyurea |
| 07 | F/32 | Mar. 4, 2002 | Myeloid blast crisis | 20.1 | 82 | Imatinib stopped |
| | | | | | | 2 weeks prior |
| 08 | M/50 | Jan. 3, 2003 | Myeloid (M7) blast crisis^{a} | 4.4 | 80 | Imatinib 600 mg daily |
| 09 | M/31 | Mar. 16, 2006 | Myeloid blast crisis | 40.1 | 79 | Hydroxyurea |
| | | Apr. 18, 2006 | Myeloid blast crisis | 3.5 | 55 | Nilotinib |
| | | May 23, 2006 | Myeloid blast crisis | 55.3 | 80 | Nilotinib + hydroxyurea |
| 10 | F/57 | Jun. 26, 2006 | Myeloid blast crisis | 0.9 | 5 | Dasatinib |
| | | Aug. 10, 2006 | Myeloid blast crisis | 7.9 | 32 | None |
| 11 | F/66 | Dec. 1, 2004 | Myeloid blast crisis | 9.3 | 43 | Imatinib 300 mg daily |
| | | | | | | stopped 1 week prior |
| 12 | M/59 | Mar. 24, 2006 | Myeloid blast crisis | 51.5 | 74 | Dasatinib |
| 13 | M/81 | May 30, 2006 | Myeloid blast crisis | 6.0 | 77 | Imatinib 600 mg daily, |
| | | | | | | P210 BCR-ABL not detected |
| 14 | F/80 | Dec. 15, 2000 | Lymphoid blast crisis | 46.2 | 93 | Imatinib 300 mg daily |

^{a}M7: Megakaryocytic blast crisis per the French-American-British (FAB) classification
CML: chronic myelogenous leukemia; WBC: white blood cell; PB: peripherial blood

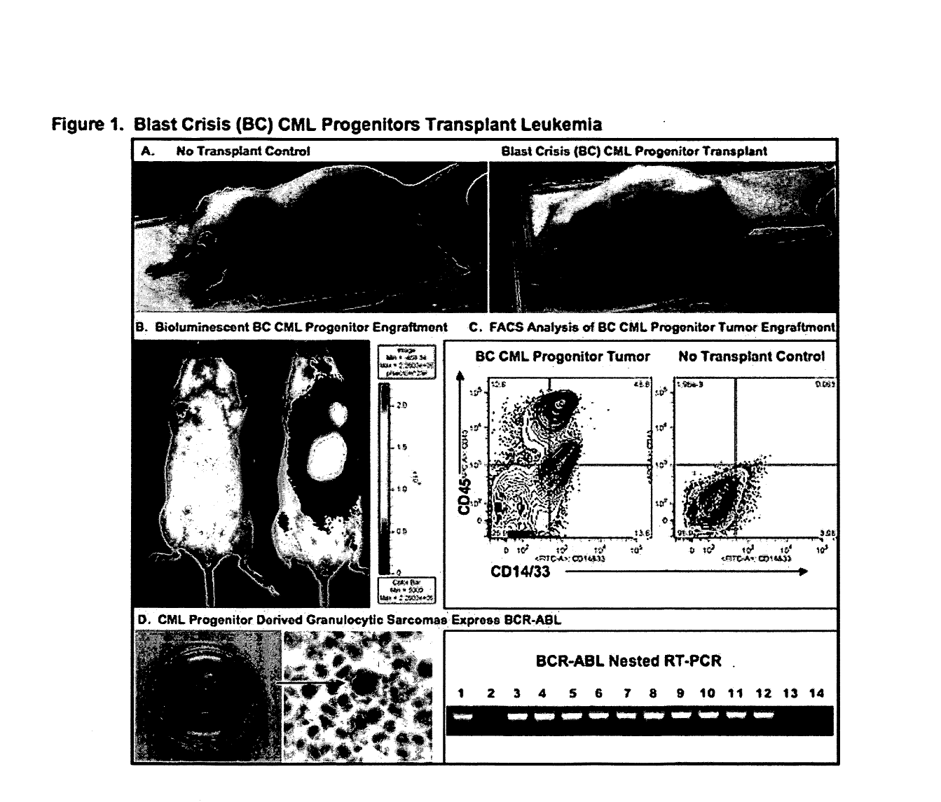

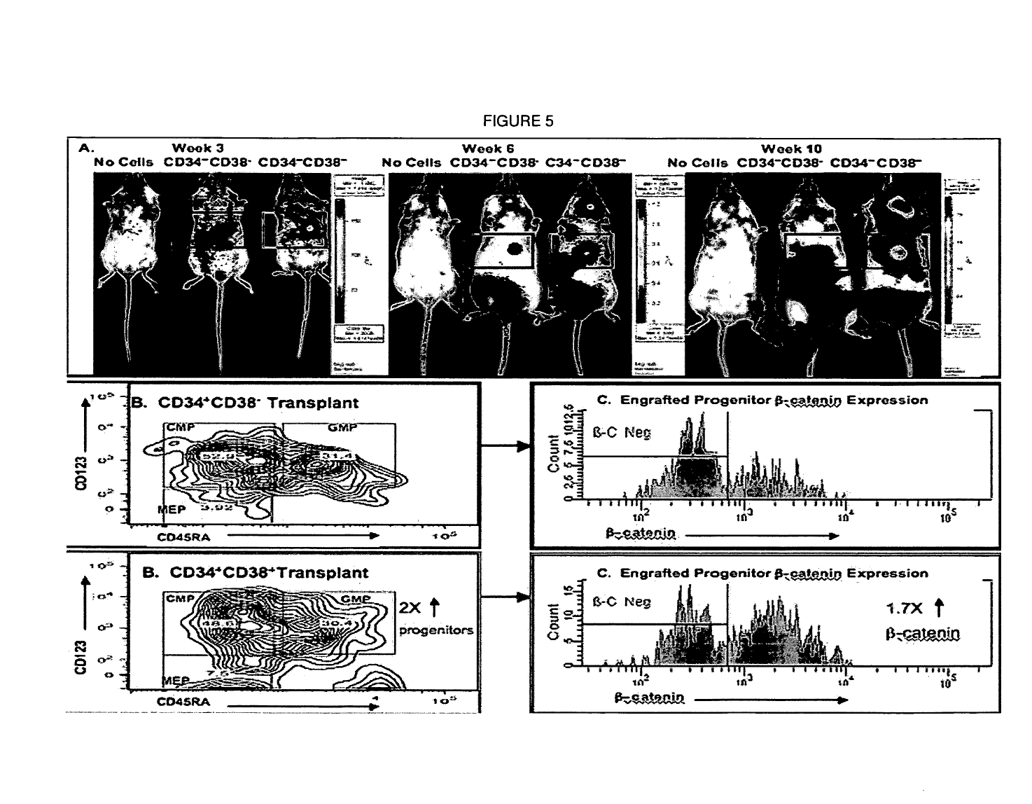

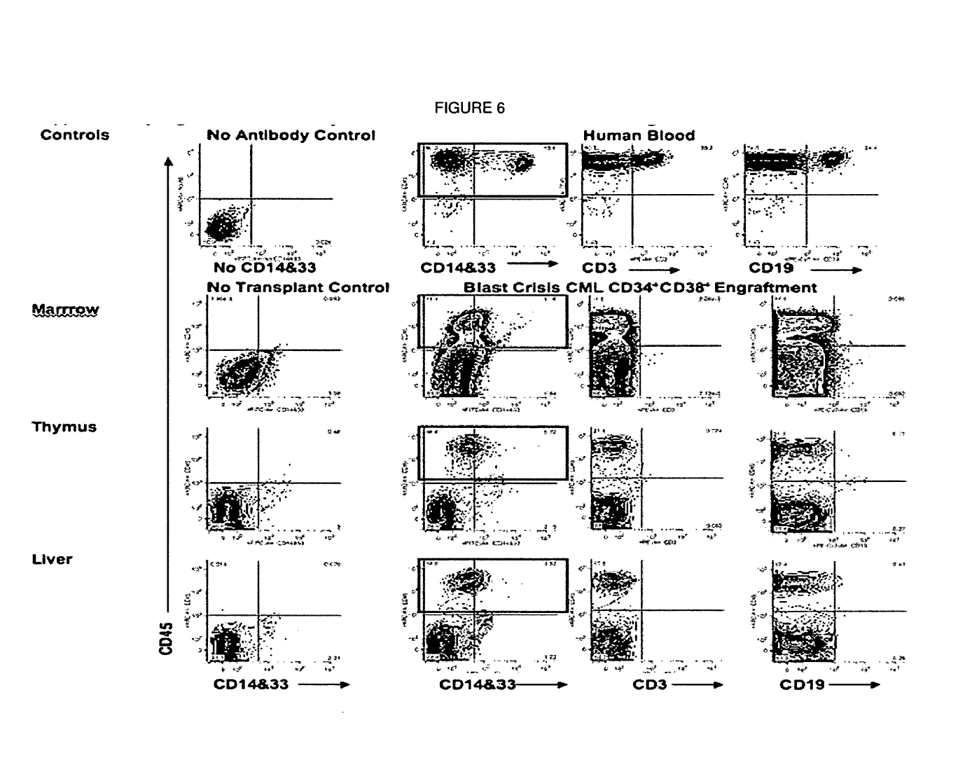

Weekly in vivo bioluminescent imaging analyses demonstrated enhanced hepatic engraftment of lentiviral luciferase transduced BC progenitors (CD34⁺CD38⁺Lin⁻) that yielded twice as many myeloid progenitors, prominent myeloid engraftment in all hematopoietic tissues and 1.7-fold higher β-catenin expression than CD34⁺CD38⁻Lin cells (FIGS. 5 and 6). Moreover, mice transplanted with lentiviral luciferase transduced BC CML CD34⁺CD38⁺Lin⁻ cells developed signs of leukemia including wasting, piloerection, lethargy as well as BCR-ABL expression in all hematopoietic tissues and bioluminescent subcutaneous BCR-ABL+ tumors, typical of granulocytic sarcomas (FIG. 1).

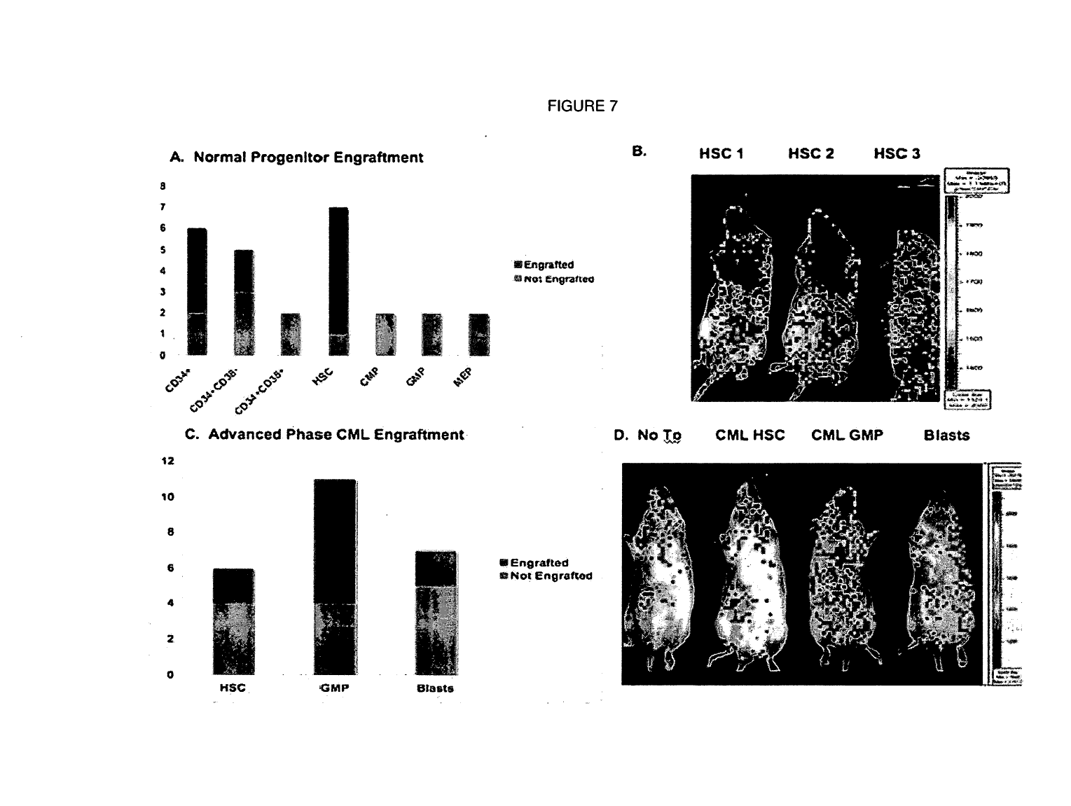

Because the CD34⁺CD38⁺Lin⁻ fraction of BC CML blood and marrow is composed primarily of GMP, we examined whether GMP also produced leukemia. Myeloid BC GMP promoted engraftment more frequently than BC HSC and blasts (FIG. 7). Normal human HSC populations also engrafted long-term, while normal committed progenitors, including GMP, did not (FIG. 7). These results suggested that self-renewing LSC were enriched within the GMP fraction of myeloid BC CML.

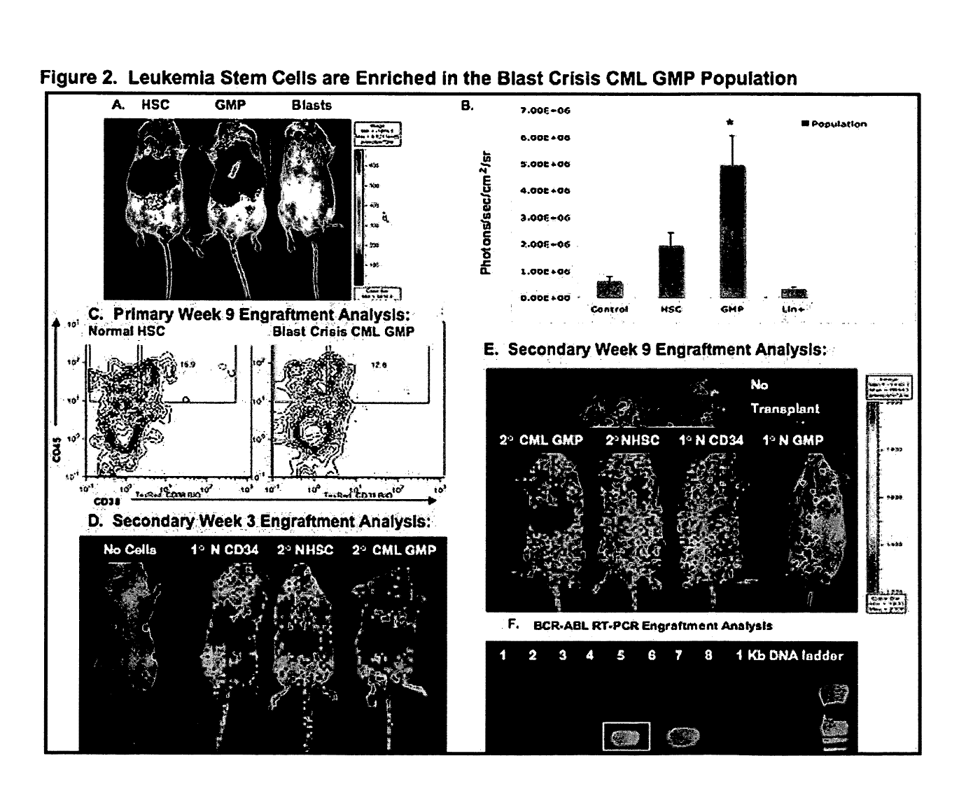

To assess the kinetics of LSC versus normal HSC engraftment, weekly in vivo bioluminescent imaging was performed following transplantation of lentiviral luciferase transduced cells. In these studies, BC GMP (LSC) showed significantly greater bioluminescent engraftment capacity than BC HSC or blasts compared with no transplant controls (FIG. 2). Moreover, BC GMP engraftment kinetics paralleled those of normal HSC (FIG. 2). Self-renewal potential of BC GMP was assessed by serially transplanting human CD45⁺ cells derived from hematopoietic tissues of BC GMP transplanted RAG2^{-/-}γ_{c}^{-/-} mice. Serial transplantation capacity of BC GMP was compared with that of BC HSC and blasts as well as normal GMP and HSC. Both normal HSC and BC GMP demonstrated serial engraftment potential absent in more committed normal progenitors (FIG. 2; FIG. 7). These experiments suggested that BC GMP had aberrantly gained self-renewal capacity while retaining BCR-ABL expression in vivo and thus, behaved like LSC (FIG. 2).

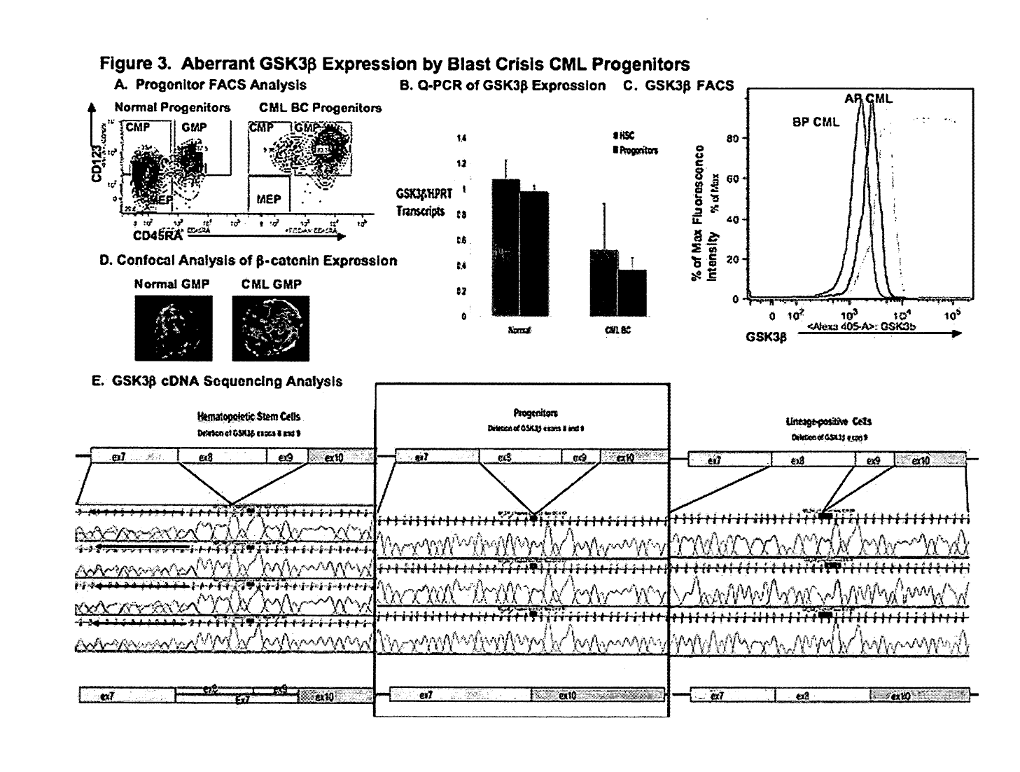

Research has demonstrated activation of components of the Wnt/β-catenin self-renewal pathway during progression to BC CML. Thus, we sought molecular mechanisms driving β-catenin activation and expansion of the BC GMP pool by quantitative RT-PCR analysis of key Wnt/β-catenin pathway gene transcript levels. This analysis revealed a significant decrease in full-length GSK3β transcripts in BC CML progenitors compared with their normal counterparts (FIG. 3).

Moreover, FACS analysis demonstrated decreased GSK3β protein expression by CML progenitors during progression from CP and accelerated phase (AP) to BC (FIG. 3). Unlike in normal GMP, confocal fluorescence microscopic analysis revealed that GSK3β-depleted BC GMP had high levels of nuclear activated β-catenin (FIG. 3). To elucidate the genetic and epigenetic events responsible for decreased GSK3β expression in BC CML and if other Wnt pathway mediators were aberrantly regulated, 49 normal (Table 2A) and 51 CML (Table 2B) progenitor populations were analyzed by genomic DNA and/or cDNA sequencing. Direct DNA sequencing was utilized to identify genomic DNA mutations in critical Wnt/β-catenin signaling pathway genes including β-catenin, GSK3β, axin 1, APC, cyclin D1, LEF-1 and c-myc.

TABLE 2A
GSK3β, APC, Axin 1, β-catenin, c-myc, Cyclin D1
and LEF-1 Genomic and cDNA Sequencing Analysis:
Normal Cord Blood (CB), Peripheral Blood (PB)
and Bone Marrow (BM) Populations
N = 49

| | Sample | Subpopulation | gDNA | cDNA | % Transcripts |
|---|---|---|---|---|---|
| 1 | Normal CB1 | HSC | NVD | NA | |
| 2 | Normal CB1 | HSC | NVD | NA | |
| 3 | Normal CB1 | HSC | NVD | NA | |
| 4 | Normal CB1 | HSC | NVD | NA | |
| 5 | Normal CB1 | GMP | NVD | NA | |
| 6 | Normal CB1 | GMP | NVD | NA | |
| 7 | Normal CB1 | GMP | NVD | NA | |
| 8 | Normal CB1 | GMP | NVD | NA | |
| 9 | Normal CB2 | HSC | NVD | NA | |
| 10 | Normal CB2 | HSC | NVD | NA | |
| 11 | Normal CB2 | HSC | NVD | NA | |
| 12 | Normal CB2 | HSC | NVD | NA | |
| 13 | Normal CB3 | HSC | NA | GSK3β exon 9 del | 50 |
| 14 | Normal PB1 | HSC | NVD | NA | |
| 15 | Normal PB1 | 34⁺38⁺ | NVD | NA | |
| 16 | Normal PB1 | GMP | NVD | NA | |
| 17 | Normal PB1 | Lin⁺ | NVD | NA | |
| 18 | Normal PB2 | HSC | NVD | NA | |
| 19 | Normal PB2 | 34⁺38⁺ | NVD | NA | |
| 20 | Normal PB2 | GMP | NVD | NA | |
| 21 | Normal PB3 | 34⁺38⁻ | NA | GSK3β exon 9 del | 100 |
| 22 | Normal PB3 | 34⁺38⁺ | NA | GSK3β exon 9 del | 50 |
| 23 | Normal PB3 | GMP | NA | GSK3β exon 9 del | 100 |
| 24 | Normal PB3 | Lin⁺ | NA | GSK3β exon 9 del | 30 |
| 25 | Normal PB4 | 34⁺38⁺ | NA | GSK3β exon 9 del | 100 |
| 26 | Normal PB4 | Lin⁺ | NA | GSK3β exon 9 del | 30 |
| 27 | Normal PB5 | 34⁺38⁺ | NA | GSK3β exon 9 del | 50-100 |
| 28 | Normal PB5 | CMP | NA | GSK3β exon 9 del | 50-100 |
| 29 | Normal PB5 | GMP | NA | GSK3β exon 9 del | 100 |
| 30 | Normal PB5 | MEP | NA | GSK3β exon 9 del | 50 |
| 31 | Normal PB5 | 34⁺38⁻ | NA | GSK3β exon 9 del | 100 |
| 32 | Normal PB5 | 34⁺38⁺ | NA | GSK3β exon 9 del | 100 |
| 33 | Normal PB5 | Lin⁻ | NA | GSK3β exon 9 del | 30 |
| 34 | Normal PB6 | 34+38− | NA | GSK3β exon 9 del | 100 |
| 35 | Normal PB6 | 34+38+ | NA | GSK3β exon 9 del | 50 |
| 36 | Normal PB6 | GMP | NA | GSK3β exon 9 del | 100 |
| 37 | Normal PB6 | Lin+ | NA | GSK3β exon 9 del | 30 |
| 38 | Normal PB7 | 34+38+ | NA | GSK3β exon 9 del | 100 |
| 39 | Normal PB7 | Lin+ | NA | GSK3β exon 9 del | 30 |
| 40 | Normal PB8 | CD34-enriched | NA | GSK3β exon 9 del | 20 |
| 41 | Normal PB8 | CD34-negative | NA | GSK3β exon 9 del | 20 |
| 42 | Normal PB9 | CD34 enriched | NA | GSK3β exon 9 del | 20 |
| 43 | Normal PB9 | CD34-negative | NA | GSK3β exon 9 del | 20 |
| 44 | Normal PB10 | CD34-enriched | NA | GSK3β exon 9 del | 20 |
| 45 | Normal PB10 | CD34-negative | NA | GSK3β exon 9 del | 20 |
| 46 | Normal BM1 | HSC | NA | GSK3β exon 9 del | 50 |
| 47 | Normal BM1 | GMP | NA | GSK3β exon 9 del | 50 |
| 48 | Normal BM2 | HSC | NA | APC E1317Q | 50 |
| 49 | Normal BM2 | GMP | NA | APC E1317Q | 50 |

NVD, no variant detected; NA, not analyzed; Del, deletion; HSC, hematopoietic stem cell; Lin⁺, lineage-positive gDNA genomic DNA

TABLE 2B
GSK3β, APC, Axin 1, β-catenin, c-myc, Cyclin D
1 and LEF-1 Genomic and cDNA Sequencing Analysis:
CML Chronic Phase (CP), Accelerated Phase (AP), Blast
Phase (BP) Peripheral Blood (PB) and Bone Marrow (BM)
N = 51

| 1 | CML CP1 PB | HSC | NVD | NA | |
|---|---|---|---|---|---|
| 2 | CML CP1 PB | 34⁺38⁺ | NVD | NA | |
| 3 | CML CP1 PB | Lin⁺ | NVD | NA | |
| 4 | CML CP2 PB | 34⁺38⁻ | NA | GSK3β exon 9 del | 100 |
| 5 | CML CP2 PB | 34⁺38⁺ | NA | GSK3β exon 9 del | 100 |
| 6 | CML CP2 PB | Lin⁺ | NA | GSK3β exon 9 + 11 del | 100 |
| 7 | CML CP3 PB | 34⁺38⁻ | NA | GSK3β exon 8 + 9 del | 100 |
| 8 | CML CP3 PB | 34⁺38⁺ | NA | •GSK3β exon 8 + 9 del | 100 |
| 9 | CML CP3 PB | Lin⁺ | NA | NA | |
| 10 | CML CP4 PB | 34⁺38⁻ | NA | GSK3β exon 9 del | 100 |
| 11 | CML CP4 PB | 34⁺38⁻ | NA | NA | |
| 12 | CML CP4 PB | Lin⁻ | NA | GSK3β exon 9 del | 100 |
| 13 | CML AP1 BM | HSC | NVD | NA | |
| 14 | CML AP1 BM | HSC | NVD | NA | |
| 15 | CML AP1 BM | HSC | NVD | NA | |
| 16 | CML AP1 BM | HSC | NVD | NA | |
| 17 | CML AP1 BM | GMP | NVD | NA | |
| 18 | CML AP1 BM | GMP | NVD | NA | |
| 19 | CML AP1 BM | GMP | NVD | NA | |
| 20 | CML AP1 BM | GMP | NVD | NA | |
| 21 | CML BP1 BM | HSC | NA | GSK3β exon 8 + 9 del | 50 |
| 22 | CML BP1 BM | GMP | NA | GSK3β exon 8 + 9 del | 100 |
| 23 | CML BP1 BM | Lin⁻ | NA | NVD | |
| 24 | CML BP2 BM | GMP | NA | NVD | |
| 25 | CML BP2 BM | Lin⁺ | NA | NVD | |
| 26 | CML BP3 BM | HSC | NA | NVD | |
| 27 | CML BP3 BM | GMP | NA | NVD | |
| 28 | CML BP4A PB | HSC | NVD | GSK3β exon 8 + 9 del | 50 |
| 29 | CML BP4A PB | GMP | NVD | GSK3β exon 8 + 9 del | 100 |
| 30 | CML BP4A PB | Lin⁻ | NVD | GSK3β exon 9 del | 100 |
| 31 | CML BP4B BM | HSC | NA | NA | |
| | | | | c-myc AAC > AGC N11S dbSNP: rs4645959 | |
| 32 | CML BP4B BM | GMP | NA | GSK3β exon 8 + 9 del | 100 |
| 33 | CML BP4B BM | Lin⁻ | NA | GSK3β exon 11 del | 50 |
| 34 | CML BP4C PB | HSC | NVD | GSK3β exon 9 del | 50 |
| 35 | CML BP4C PB | GMP | NVD | GSK3β exon 8 + 9 del | 500 |
| 36 | CML BP4C PB | Lin⁺ | NVD | GSK3β exon 9 del | 100 |
| 37 | CML BP5 PB | HSC | NVD | GSK3β exon 8 + 9 del | 50 |
| 38 | CML BP5 PB | CD34⁺CD38⁺ | NVD | GSK3β exon 8 + 9 del | 50 |
| 39 | CML BC5 PB | Lin⁻ | NVD | GSK3β exon 9 del | 100 |
| 40 | CML BP6 PB | CD34⁺CD36⁻ | NA | GSK3β exon 9 del | 100 |
| 41 | CML BP6 PB | CD34⁺CD36⁺ | NA | GSK3β exon 9 del | 100 |
| 42 | CML BP6 PB | Lin⁺ | NA | GSK3β exon 9 del | 100 |
| 43 | CML BP 7 BM | HSC | NVD | GSK3β exon 9 del | 60 |
| 44 | CML BP 7 BM | HSC | NA | c-myc TTT > TAT F375Y | 50 |
| 45 | CML BP 7 BM | GMP | NA | GSK3β exon 8 + 9 del | 100 |
| 46 | CML BP 7 BM | Lin⁺ | NA | GSK3β exon 5 del | 50 |
| 47 | CML BP 8 BM | HSC | NVD | NA | |
| 48 | CML BP 8 BM | GMP | NVD | NA | |
| 49 | CML BP 8 BM | Lin⁺ | NVD | NA | |
| 50 | CML lymphoid BP | HSC | NA | NVD | |
| 51 | CML lymphoid BP | Lin⁺ | NA | NVD | |

NVD no variant detected
NA not analyzed
Del deletion
HSC hematopoietic stem cell
GMP granulocyte-macrophage progenitor
CMP common myeloid progenitor
MEP megakaryocyte-erythroid progenitor
Lin⁺ lineage-positive

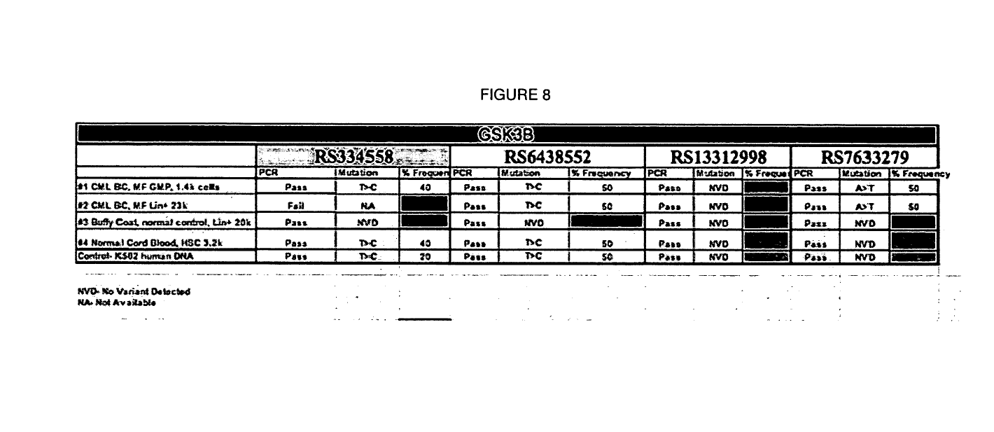

A Wnt mediator gene SNP analysis revealed three SNPs in the GSK3β gene in BC GMP previously shown to enhance alternative splicing of GSK3β (FIG. 8). This analysis suggested that these SNPs may create hematopoietic developmental stage specific novel splice acceptor sites resulting in missplicing of GSK3β. A comprehensive cDNA sequencing analysis of the same Wnt/β-catenin pathway genes revealed a novel exon 8 and 9 deleted splice isoform of GSK3β in progenitors in 57% of BC CML samples (Table 2B). While the predominant GSK3β transcript in BC GMP had an in-frame deletion of exons 8 and 9, BC HSC had an equal proportion of full length and exon 8 and 9 misspliced transcripts (Table 2B; FIG. 3).

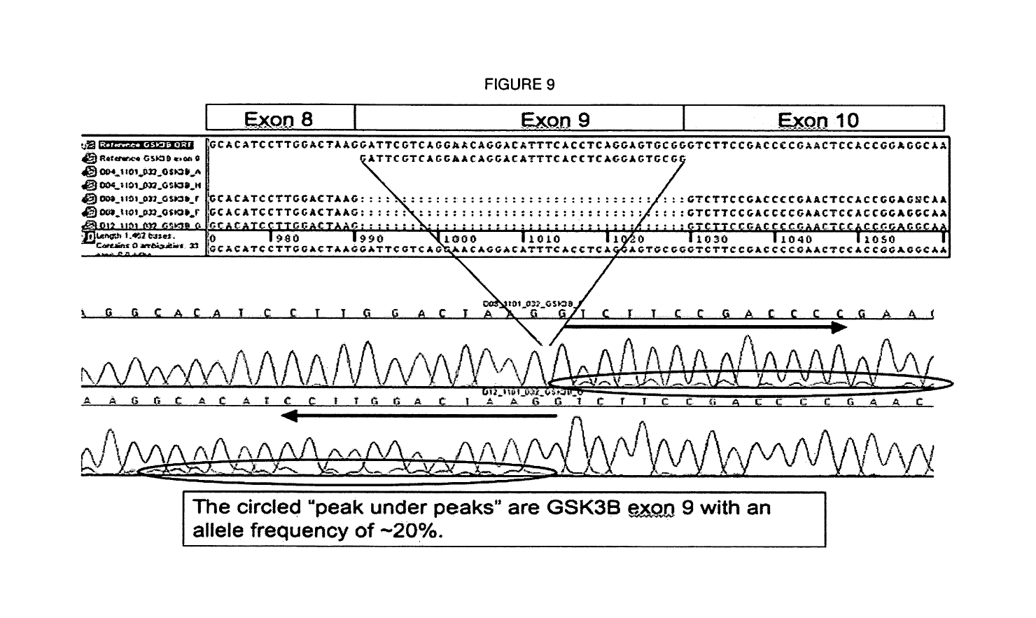

Misspliced GSK3β transcripts, lacking the FRAT and axin 2 binding domains encoded by exons 8 and 9, were not detected in CML blasts or normal (n=49) sample populations (Table 2A and B). Alternative GSK3β splice isoforms, deleted in exon 9, exon 11 or exon 9 and 11, represented 50 to 100% of GSK3β transcripts in the blasts as well as CP CML and normal peripheral blood, marrow and cord blood (Table 2A, 2B; FIG. 9). Notably, exon 9 and 11 deleted GSK3β splice isoforms were previously identified in Parkinson's Disease neuronal cells. Conversely, exon 8 and 9 truncated transcripts found in BC CML have not been described to date, suggesting that in LSC a misspliced GSK3β splice isoform predominates that cannot phosphorylate β- (or γ-) catenin.

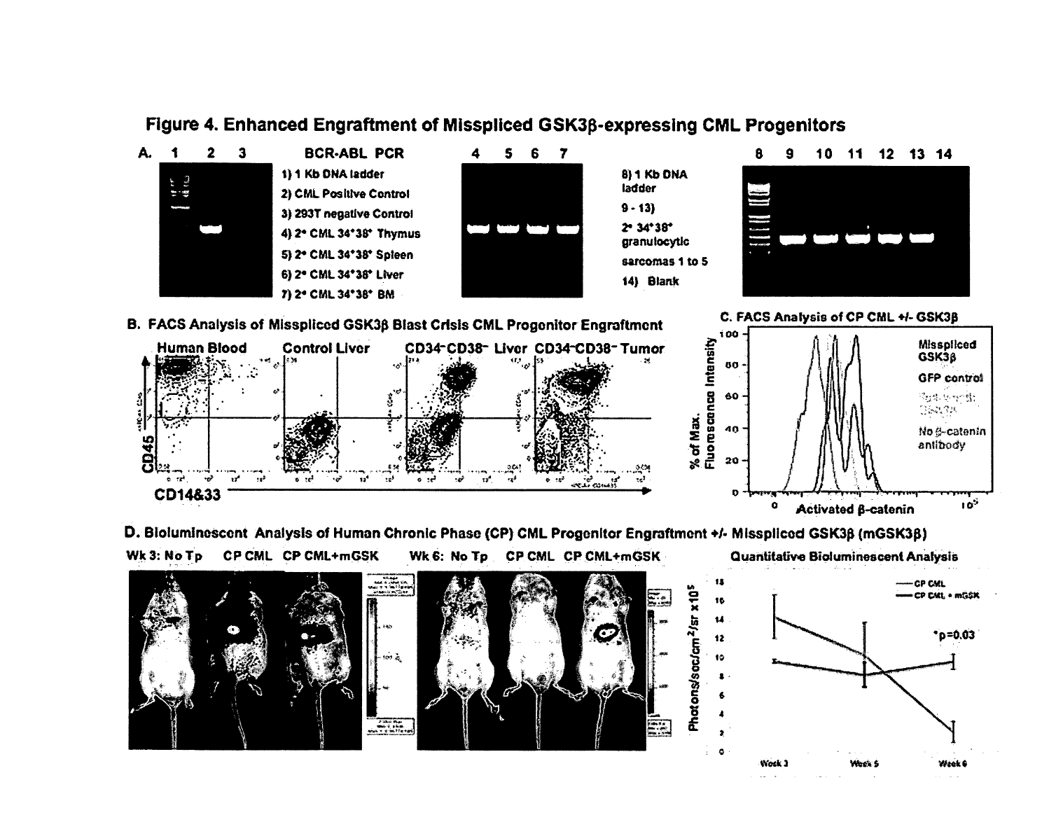

Notably, transplantation of BC progenitors expressing misspliced GSK3β produced high levels of BCR-ABL+ myeloid cell engraftment in hematopoietic tissues and tumors derived from transplanted primary and secondary recipient mice (FIG. 4). Moreover, lentiviral misspliced GSK3β transduction of CP CML progenitors led to increased levels of activated β-catenin expression while lentivirally-enforced expression of full-length GSK3β reduced β-catenin expression (FIG. 4). Finally, CP CML progenitors lentivirally co-transduced with luciferase and misspliced GSK3β had enhanced bioluminescent engraftment compared with control vector transduced progenitors (FIG. 4) suggesting that dysregulation of GSK3β through missplicing may be a key event in the evolution of LSC. These studies suggest that hematopoietic developmental stage-specific decreases in functional GSK3β, through missplicing that deletes the axin 2 and FRAT binding sites responsible for GSK3β-mediated enhanced β-catenin phosphorylation [exon 8] and subsequent degradation may represent an important mechanism triggering aberrant β-catenin activation, nuclear entry and enhanced self-renewal capacity.

We have previously shown that a master regulator of hematopoiesis, Ikaros, has hematopoietic stem, progenitor and progeny stage specific splice isoforms in mice. Single nucleotide polymorphisms (SNPs) previously predicted to create alternative splice acceptor sites responsible for generating GSK3β splice isoforms, were detected in CML LSC(CML GMP) that harbored the exon 8 and 9 deleted transcripts suggesting a possible mechanism for misspliced GSK3β transcript formation. Alternatively, these transcripts could arise through epigenetic fixation of splice isoforms, mutations in an as yet unknown splicing element or spliceosomal errors. Changes in splicing have been shown to play a functionally significant role in tumorigenesis, either by inactivating tumor suppressors or by gain of function of proteins promoting tumor development.

Cancer specific splicing events may generate novel epitopes that can be recognized by the hosts immune system as cancer specific and serve as targets for immunotherapy. In this vein, the identification of LSC specific splice isoforms of GSK3β provides a useful indicator of disease progression, and a therapeutic target for eradicating the reservoir of LSC in advanced phase CML.

### Methods

FACS Analysis and Sorting. Normal blood and marrow samples were purchased from the San Diego Blood Bank or All Cells (Berkeley, Calif.). CML blood and marrow samples were donated by Stanford University and MD Anderson Cancer Center patients according to IRB approved protocols. HSC, progenitor and Lin⁺ cells were purified via FACS as previously described.

Lentiviral Transduction and Transplantation. Equal numbers of normal or CML HSC, progenitors and Lin+ cells were FACS sorted, transduced with lentiviral vectors according to established methods and transplanted intrahepatically into neonatal RAG2^{-/-}γc^{-/-} mice. Weekly bioluminescent imaging was performed with an in vivo imaging system (IVIS 200; Caliper Inc, Hopkinton, Mass.). When moribund or at 8-12 weeks post-transplant, mice were euthanized and single cell suspensions of hematopoietic organs and tumors were analyzed for human cell engraftment via FACS. Transplanted mouse hematopoietic tissue BCR-ABL transcripts were detected by PCR with a one step RT-PCR kit (Qiagen, Valencia, Calif.). BCR-ABL product sizes for b2a2 and b3a2 were 383 bp and 458 bp, respectively.

GSK3β and β-catenin Analysis. Normal or CML CD34⁺ cells were stained with a rabbit anti-human GSK3β (#9315, Cell Signaling Technology, Danvers, Mass.) or anti-activated β-catenin monoclonal antibody (clone 8E7, Upstate Technologies, Avon, N.Y.). Quantitative RT-PCR to detect GSK3β transcripts in normal versus CML HSC and progenitors, was performed with the aid of a SYBR Greener two-Step Q-RT-PCR Kit (Invitrogen, Carlsbad, Calif., USA)

Wnt Pathway Mutation Screening. Genomic DNA mutation and cDNA analysis of β-catenin, APC, axin 1, c-myc, LEF-1, cyclin D1 and GSK3β was conducted with SURVEYOR mismatch cleavage analysis using the WAVE-HS System (Transgenomic, Gaithersberg, Md.) followed by bidirectional sequence analysis on an ABI 3100 sequencer (Applied Biosystems, Inc., Foster City, Calif.)

Statistical Analysis. Statistical analyses were performed with the aid of FloJo, Caliper and Excel software.

Flow Cytometric Analysis and Cell Sorting. Mononuclear fractions were extracted from peripheral blood or bone marrow following Ficoll density centrifugation according to standard methods. Samples were analyzed fresh or subsequent to rapid thawing of samples previously frozen in 90% FCS and 10% DMSO in liquid nitrogen. CD34⁺ cells were enriched from mononuclear fractions with the aid of immunomagnetic beads (CD34⁺ Progenitor Isolation Kit, Miltenyi Biotec, Bergisch Gladbach, Germany) and then stained with a cocktail of antibodies (CD2, 3, 4, 8, 10, 14, 19, 20, 56, +/−glycophorin A, +/−CD7, +/−CD11b) specific for terminally differentiated cells to identify the lineage positive (Lin⁺) fraction. Subsequently, cells were washed and stained with a cocktail of antibodies specific for HSC including CD34-APC, CD38-biotin detected with Streptavidin PE Cy7 and CD90 PE or a myeloid progenitor antibody cocktail consisting of CD34APC, CD38 Biotin detected with Streptavidin-PE Cy7, CD45RA-FITC and CD123-PE as previously described. Cells were analyzed and FACS purified according to established methods.

Lentiviral Production and Transduction. The lentiviral vector pHR2 was made by replacement of eGFP in pHR′tripCMV-GFPSIN by a multiple cloning site. The GFP⁻ firefly luciferase fusion (GLF) gene was subcloned from pJW.GFP-yLuc into pHR2 to generate pHR2-GLF. Lentiviral particles were prepared as previously described. The titer was determined by infection of 293T cells with several dilutions of the virus (MOI approximately 100) and analysis of GFP⁺ cells by FACS 48 hours later. Equal numbers (10³-4×10⁵) of normal or CML HSC, progenitors and Lin⁺ cells were sorted with the aid of a FACS Aria directly into 100 microliters of myelocult media (Stem Cell Technologies Inc) supplemented with cytokines (R&D Systems) including SCF (50 ng/ml), TPO (10 ng/ml), Flt3 ligand (50 ng/ml) and IL-6 (10 ng/ml) and transferred to a 96 well U bottom plate containing 100 microliters of cytokine-supplemented myelocult media per well. Cells were incubated in a 7% CO₂, 37° C. humidified incubator for 48 hours and transduction efficiency was assessed by fluorescence microscopy-mediated GFP detection. Prior to transplantation cells were centrifuged for 5 min at 300×g and resuspended in 10 microliters of myelocult media prior to transplantation. For GSK3β virus preparation, GSK3β was amplified by PCR from Plasmid (Openbiosystems Clone ID 3357620 Accession BC000251), normal peripheral blood cDNA or the GMP fraction of a BC CML BC harboring the misspliced GSK3b isoform using the following primers: FW primer: ctagtctagaaaggtgattcgcgaagagag; Rev primer: tcccccgggactggtggtttttcctgtgc. PCR products were then digested with XbaI and ClaI and cloned into a lentiviral backbone vector harboring RES GFP under the control of a CMV promoter.

Transplantation and Engraftment Analysis. Immunocompromised (RAG2^{-/-}γ_{c}^{-/-}) mice deficient in T, B and NK cells were bred and maintained in a barrier facility in the Moores UCSD Cancer Center vivarium. Neonatal RAG2^{-/-}γ_{c}^{-/-} mice were transplanted within 48 hours of birth intrahepatically with equal numbers (10³-4×10⁵ cells/mouse) of HSC, progenitors or Lin⁺ cells in a 10 μl volume with the aid of a Hamilton syringe. In 8 experiments (n=30 mice) involving normal marrow or cord blood and 12 experiments involving blast crisis CML (n=43 mice), neonatal RAG2^{-/-}γ_{c}^{-/-} mice were transplanted with equivalent numbers (10³ to 4×10⁵) of luciferase⁻GFP transduced HSC, progenitors or Lin⁺ cells.

Bioluminescent imaging, used to monitor engraftment kinetics, started at week 3 post⁻transplant and continued weekly until the time of sacrifice. Briefly, this involved intraperitoneal administration of 200 μl of luciferin (Caliper Inc) and bioluminescent imaging of isofluorane anaesthetized mice with the aid of a highly sensitive in vivo imaging system (IVIS 200; Caliper Inc). Bioluminescent imaging of non-transplanted mice served as a negative control for each experiment. When moribund or at 8-12 weeks post⁻transplant, mice were euthanized with inhaled CO₂. Single cell suspensions of hematopoietic organs including blood, liver, spleen, marrow and thymus as well as tumors were analyzed for the presence of human cells via FACS. Following ammonium citrate lysis of red blood cells for 10 min at 4° C., an anti-human Fcγ receptor blocking antibody (Miltenyi, Human CD34 Progenitor Isolation kit) was added (dilution of 1/50) and anti⁻mouse Fcγ receptor (CD16/CD32, BD Pharmingen) for 30 min. Cells were then stained with mouse monoclonal antibodies (Invitrogen) specific for human CD45 (CD45⁻ APC), CD33 and CD14 (both conjugated to FITC), CD19 (biotinylated and detected with streptavidin⁻PE⁻Cy7) and CD3 (CD3⁻PE, BD Pharmingen). Hematopoietic organs from non⁻transplanted mice and normal human peripheral blood served as negative and positive controls, respectively. Mice with greater than 1% human cells were deemed to be engrafted. Engrafted human CD45⁺ cells were sorted into RLT buffer (Qiagen RNeasy) and RNA extracted with the RNeasy kit as previously described for BCR-ABL analysis. In secondary transplantation experiments, human cells from marrow, spleen, liver and tumors were sorted into 100 μl of myelocult media for intrahepatic transplantation into neonatal RAG2^{-/-}γ_{c}^{-/-} mice. Following secondary transplantation experiments, mice showed signs of leukemia including wasting, piloerection, ataxia, and granulocytic sarcomas as early as week 3 earlier and were euthanized when moribund.

Nested p210 BCR-ABL RT-PCR. Total RNA was isolated using a RNeasy Mini Kit (Qiagen, Valencia, Calif.)₂ and quantified using a NanoDrop® ND⁻1000 Spectrophotometer (Wilmington, Del.). In primary transplantation experiments, preparation of cDNA from 2 μg of total transplanted mouse tissue RNA was carried out using a “High Capacity cDNA Archive Kit” (ABI, Foster City, Calif.). Then, p210 amplification was performed with 5 μl cDNA and 21 μl of reaction mixture consisting of 1× Buffer II (ABI, Foster City, Calif.), 2.5 mM MgCl₂, 0.2 mM dNTP's, NBI⁺ (4 ng/μl), ABL3-(4.2 ng/μl) and 5 U/reaction of AmpliTaq Gold (ABI, Foster City, Calif.). The second round of p210 BCR⁻ABL amplification was performed with 1 μl of amplified product and 20 μl of reaction mixture consisting of 1× Buffer II (ABI, Foster City, Calif.), 1.75 mM MgCl₂, 0.2 mM dNTP's, 4.4 ng/μl CA3⁻, 3.4 ng/μl B2A and 5 U/reaction of AmpliTaq Gold (ABI, Foster City, Calif.). In subsequent transplantation experiments, a one-step RT-PCR kit (Qiagen Inc) was used with the BCR-ABL same primers. Electrophoresis of amplified products was performed on a 1.5% agarose gel with expected product sizes for b2a2 and b3a2 of 383 bp and 458 bp respectively.

GSK3β and β-catenin FACS Analysis. Normal and CML CD34⁺ enriched cells from CP, AP and BC CML peripheral blood or marrow were first stained with lineage antibodies and then stained with either hematopoietic stem cell or progenitor cocktails of antibodies followed by fixation with 0.8% paraformaldehyde for 10 min. Cells were then washed by centrifugation at 1,200 rpm for 5 min and resuspended in HBSS with a rabbit monoclonal antibody specific for the first 20 N-terminal amino acids of human GSK3β (#9315, Cell Signaling Technology, Danvers, Mass.) diluted 1:50 in 0.15% Saponin (TCI America, Portland, Oreg.) and 10% goat serum (Sigma-Aldrich, Saint Louis, Mo.) for 45 min at 4° C. Cells were then washed and stained with an Alexa 405-conjugated goat anti-rabbit antibody (Molecular Probes, Eugene, Oreg.) diluted 1:400 for 45 min at 4° C. to detect the GSK3β expressing cells. Control stain with only the Alexa-405 goat anti-rabbit antibody was also done to evaluate the unspecific background staining. Mean fluorescence intensity was analyzed with the aid of a FACS Aria and FloJo software. FACS analysis of activated β-catenin expression was performed in a similar manner on single cell suspensions of livers from mice transplanted with blast crisis CML CD34⁺CD38⁻Lin⁻ or CD34⁺CD38⁺Lin⁻ cells with the proviso that the β-catenin antibody (clone 8E7, Upstate) was directly labeled (Xenon kit, Invitrogen) with Alexa-405 according to the manufacturer's specification.

GSK3β Quantitative RT-PCR. Hematopoietic stem and progenitor (1,000 to 50,000) cells were sorted directly into RLT Buffer and total RNA was isolated using RNeasy Micro Kit (Qiagen, Valencia, Calif. USA), according to manufacturer's protocol. A SYBR Greener two-Step Q-RT-PCR Kit for the iCycler (Invitrogen, Carlsbad, Calif., USA) was then used to synthesize cDNA and assess GSK3β relative transcript quantities according to the manufacturer's protocol. Briefly, 8 μl of 4 to 75 ng/μl of RNA were mixed with RT Reaction Mix and RT Enzyme Mix and incubated at 25° C. for 10 min, followed by 50° C. for 30 min and finally 85° C. for 5 min. The tubes were then chilled and 1 μl of RNase H was added to the reaction followed by a 20 min incubation at 37° C. The quantitative PCR (Q⁻PCR) reaction was performed in duplicate using 2 μl of the template in 25 μl reaction volume containing SYBR Greener Super Mix and 0.4 μM of each forward and reverse primer. The following primer pairs flanking GSK3β exons 9 and 10 were used: GSK3β Forward: gacatttcacctcaggagtgc GSK3β Reverse: gttagtcgggcagttggtgt HPRT Forward: cgtcttgctcgagatgtgatg HPRT Reverse: tttatagccccccttgagcac for all reactions. Relative values of transcripts were determined according to a standard curve. GSK3β values were then normalized to HPRT values.

Wnt Mutation Screening. Wnt/β-catenin Signaling Pathway Mutation Analysis. Targeted Wnt/β-catenin pathway regulator genomic DNA mutation and cDNA analysis was performed on normal or CML FACS-sorted CD34⁺CD38⁻Lin⁻, CD34⁺CD38⁺Lin⁻, HSC, GMP, and lineage-positive cells. Wnt/β-catenin pathway genes analyzed included β-catenin, GSK3β, adenomatous polyposis coli (APC), axin 1, c-myc, cyclin D1 (CCND1) and LEF-1. Sample preparation and RNA Extraction Total RNA was extracted from snap-frozen cells and colonies using either the RNeasy® Mini Protocol (Qiagen, Germantown, Md.) or TRIZOL® reagent (Invitrogen, Carlsbad, Calif.) according to the instructions of the manufacturers. All samples were quantified using the NanoDrop® ND⁻1000 Spectrophotometer (Wilmington, Del.) and resuspended at working concentrations of 100 ng/μl in RNase free water.

Reverse Transcription and Polymerase Chain Reaction. (RT-PCR) SuperScript III First⁻Strand Synthesis System for RT-PCR with Platinum® Taq (Invitrogen, Carlsbad, Calif.) and 50 ng of random hexamers according to the manufacturer's recommendations in individual tubes for each RNA sample with 200 U of the SuperScript III RT enzyme. PCR was performed using 100 ng of the cDNA as template in separate 50 μl reactions which consisted of final concentrations of 1.25 U of HotMaster Taq DNA Polymerase (Eppendorf), HotMaster Taq Buffer with 2.5 mM Mg2⁺ (25 mM Tris-HCL pH 8.0, 35 mM KCL, 0.1 mM EDTA, 1 mM DDT, 50% glycerol, 0.5% Tween20, 0.5% IGEPAL CA-630 and stabilizers), 2 mM of each dNTP, 0.2 μM of each sense and anti-sense primer. Reverse transcription and PCR cycling steps were carried out in a MJ Research Dyad thermocycler. The conditions for first strand cDNA synthesis were 65° C. for 5 min, followed by 25° C. for 10 min and 50° C. for 50 min. Reactions were terminated at 85° C. for 5 min. PCR amplification of the newly synthesized cDNA performed by 35 cycles of denaturation (94° C., 15 sec), annealing (55° C., 30 sec), and extension (68° C., 2 min) followed by a final extension step of 1 cycle at 68° C. for 5 min.

GSK3β outer PCR primers used in the PCR amplifications were SEQ ID NO:3 (forward) 5′ aaggtgattcgcgaagagag ⁻3′ and SEQ ID NO:4 (reverse) 5′ actggtggtttttcctgtgc⁻3′ These primers amplify a 1343 by cDNA product that contains the open reading frame (ORF) of GSK3β. Multiple nested inner primers were used to produce full double stranded sequencing coverage of the entire ORF.

Sequence Scanning. Sequence analysis of the β-catenin, APC, axin 1, c-myc, LEF-1, cyclin D1 and GSK3β cDNA PCR products were conducted with SURVEYOR mismatch cleavage analysis using the WAVE-HS System (Transgenomic, Omaha, Nebr.) to detect sequence anomalies such as mutation and unusual splice forms. Aliquots of PCR product (3-15 μl) were scanned for mutations by Surveyor mismatch cleavage, and identified with bidirectional sequence analysis on an ABI 3100 sequencer using BigDye V3.1 terminator chemistry (Applied Biosystems, Inc., Foster City, Calif.). For semi-quantitative determination of mutant and normal allele frequencies, relative peak areas of Surveyor mismatch cleavage products were determined after normalization and comparison to reference controls using the WAVE Navigator software.

DNA sequencing and analysis Primers: SEQ ID NO:5 GSK3B ex5 For tgagtgccagtaatccatagca SEQ ID NO:6 GSK3B ex5 Rev—cacagctgcactagtttatacttcttg SEQ ID NO:7 GSK3B ex6 For—tttttgcagagcaaggtgat SEQ ID NO:8 GSK3B ex6 Rev—aagcagtgcctggaaagtataa SEQ ID NO:9 GSK3B ex7 For—tggtctggtttcctctttgg SEQ ID NO:10 GSK3B ex7 Rev—gaagcaatccaaggtcacgt SEQ ID NO:11 GSK3B ex8 For—catgagctttgggggacata SEQ ID NO:11 GSK3B ex8 Rev—agtacactgcccttaccttgc SEQ ID NO:12 GSK3B ex9 For—ccccacaccatggatttct SEQ ID NO:13 GSK3B ex9 Rev—gaatcacccaagaggctgac SEQ ID NO:14 GSK3B ex10 For—gaaggtcctttcccaaatgc SEQ ID NO:15 GSK3B ex10 Rev—tgtccgtttttgtcctccac SEQ ID NO:16 GSK3B ex11 For—gattacaggcgtgagccact SEQ ID NO:17 GSK3B ex11 Rev—cttgttggtcaggctggtct.

PCR products were obtained using the above-mentioned primers, amplifying the corresponding exon and splice junctions. Excess PCR primers were removed from 10 μL of PCR product using the Ampure PCR Purification system (Agencourt, Beverly Mass.). Purified product was eluted in 30 μL of de⁻ionized water. Standard reaction chemistry using BigDye v. 3.1 and cycle sequencing were used as per the manufacturer's recommendations. Cycle sequencing products were purified using CleanSeq reagents (Agencourt, Beverly Mass.). Purified sequencing products were eluted in 40 μL of 0.01 μM EDTA and 30 μL was run on an ABI 3100 Genetic Analyzer. DNA sequence was analyzed using ABI Sequence Analysis v3.7, Sequencher (GeneCodes, Ann Arbor, Mich.), and Mutation Surveyor (Softgenetics, State College, Pa.). The reference GSK3β (NM_{—}002093) sequence was downloaded from the UCSC Genome Bioinformatics webpage using the March 2006 assembly.

Lentiviral GSK3β Transduction Experiments. GSK3β was amplified by PCR from Plasmid (Openbiosystems Clone ID 3357620 Accession BC000251) or peripheral blood cDNA from a normal donor or the GMP fraction of a BC CML patient using the following primers: SEQ ID NO:18 Forward primer: ctagtctagaaaggtgattcgcgaagagag SEQ ID NO:19 Reverse primer: tcccccgggactggtggtttttcctgtgc PCR products were then digested with XbaI and ClaI and cloned into a lentivirus backbone harboring RES GFP under the control of a CMV promoter for in vitro and in vivo assays. For these assays, chronic phase CML peripheral blood progenitor (CD34⁺CD38⁺Lin⁻) cells were FACS-ARIA sorted into 96 well plates containing Myelocult media (Stem Cell Technologies Inc) supplemented with Flt3 ligand (3 μg/ml), Interleukin-6 (1 μg/ml), stem cell factor (4 μg/ml) and thrombopoietin (0.625 μg/ml). Sorted CML progenitor cells (2-4×10⁴ cells/well) were transduced with lentiviruses expressing full length GSK3β (GSK-Full) or GSK3β deleted in exon8 and 9 (GSK del) as well as lentiviral luciferase-GFP to permit bioluminescent imaging of engraftment in transplanted mice (2-4×10⁴/mouse; n=8). Progenitor cells were washed 48 hours after transduction and transplanted intrahepatically into neonatal RAG2^{-/-}γ_{c}^{-/-} mice.

For in vitro assays, CD34⁻ enriched cells from CML peripheral blood were plated (10⁴-10⁵ cells/well) in a 96 well plate with Myelocult media (Stem Cell Technologies Inc) supplemented with Flt3 ligand (3 μg/ml), Interleukin-6 (1 μg/ml), Stem Cell Factor (4 μg/ml) and thrombopoietin (0.625 μg/ml). Lentiviruses expressing full length GSK3β (GSK-Full), GSK3β-deleted in exons 8 and 9 (GSK del) or luciferase-GFP were then added to each well. Cells were harvested 7 days after transduction for β-catenin FACS analysis performed as described above.

Progression of blast crisis (BC) CML is associated with an expansion of the GMP compartment compared with normal blood and marrow samples and a relative decrease in megakaryocyte-erythroid progenitors (MEP) and common myeloid progenitors (CMP). There was a significant difference in GSK3β transcript levels between normal and BC CML progenitors. There is a decrease in GSk3β protein expression with progression to blast crisis. Normal granulocyte-macrophage progenitors (GMP) had little activated β-catenin. Conversely, BC CML GMP, with the GSK3β misspliced transcript, had high levels of nuclear activated β-catenin. Blast crisis CML HSC had demonstrable misspliced exon 8 and 9 deleted GSK3β transcripts. The sequence shows clean exon 7 sequence directly followed by exon 10 sequence, indicating a complete deletion of exons 8 and 9. A deletion of GSK3β exon 9 is also detectable in normal samples.

This is the first description of epigenetic dysregulation of GSK3b expression in cancer and may be used to predict progression of cancer to a more advanced form based on FACS analysis. Furthermore, detection of decreased axin 2 transcripts may have prognostic value.

These data demonstrate although that chronic phase CML is initiated by BCR-ABL expression in an HSC clone, progression to blast crisis can include missplicing of GSK3β in GMP LSC, enabling unphosphorylated β-catenin to participate in LSC self-renewal. Missplicing of GSK3β represents a unique mechanism for the emergence of blast crisis CML LSC, is a diagnostic indicator of disease progression and represents a novel therapeutic target in blast crisis CML. As the first description of GSK3β dysregulation in cancer, this research provides a focal point for investigating whether GSK3β missplicing also triggers β-catenin activation in other CSC.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method of screening a cancer, the method comprising:
analyzing a cancer cell sample for the presence of a splice variant of GSK3β.

2. The method of claim 1, wherein the cancer is a human cancer.

3. The method of claim 1, wherein the cancer is a leukemia.

4. The method of claim 2, wherein the leukemia is a myelogenous leukemia.

5. The method of claim 1, wherein the GSK3β splice variant has a deletion of exon 8.

6. The method of claim 5, wherein the splice variant further comprises a deletion of exon 9.

7. The method of claim 1, comprising the steps of contacting a biological sample comprising mRNA or a nucleic acid derived therefrom from said cancer with at least one probe that specifically binds to GSK3β.

8. The method of claim 7, wherein the probe binds to a junction of GSK3β exon 7 and exon 10.

9. The method of claim 7, wherein the sequences between exon 7 and exon 10 of GSK3b are amplified.

10. The method of claim 7, wherein the probe binds to a sequence within exon 8 of GSK3b and a decrease in hybridization compared to a control is indicative of the presence of the splice variant.

11. The method of claim 1, comprising the steps of contacting a biological sample comprising protein from said cancer with at least one agent that specifically binds to GSK3β.

12. The method of claim 11, wherein the agent is an antibody.

13. The method of claim 12, wherein the antibody is specific for the junction of exon 7 and exon 10 in GSK3β

14. The method according to claim 1, wherein said analyzing is performed in vivo.

15. The method according to claim 1, wherein said analyzing is performed in vitro.

16. A kit for use in the method set forth in claim 1.

17. A non-human mammal comprising:
exogenous xenogeneic leukemia cells; wherein said leukemia cells express a splice variant of GSK3β.

18. The mammal of claim 17, wherein the leukemia is a myelogenous leukemia.

19. The mammal of claim 18, wherein the GSK3β splice variant has a deletion of exons 8 and 9.

20. The mammal of claim 17, wherein the leukemia cells are human CD34⁺CD38⁺Lin⁻ cells.

21. The mammal of claim 20, wherein the human cells are transfected with a nucleic acid construct encoding GSK3b Δ exons 8-9.

22. The mammal of claim 17, wherein said animal is an immunocompromised mouse.

23. The mammal of claim 17, wherein the leukemia cells comprise a detectable bioluminescent marker.

24. A method for screening a candidate therapy for efficacy in treatment of a cancer, the method comprising:
transferring a cell population comprising human leukemia cells expressing GSK3b Δ exons 8-9 into an immunocompromised non-human mammal host;
treating said animals with said candidate therapy;
evaluating the human cells present in said animal.


SEQUENCE LISTING (20 sequences)

SEQ ID NO: 1 - DNA, 1621 bp, Human
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatatt

SEQ ID NO: 2 - protein, 433 aa, Human
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 3 - DNA, 20 bp, Artificial Sequence
  Primer
aaggtgattcgcgaagagag

SEQ ID NO: 4 - DNA, 20 bp, Artificial Sequence
  Primer
actggtggtttttcctgtgc

SEQ ID NO: 5 - DNA, 22 bp, Artificial Sequence
  Primer
tgagtgccagtaatccatagca

SEQ ID NO: 6 - DNA, 27 bp, Artificial Sequence
  Primer
cacagctgcactagtttatacttcttg

SEQ ID NO: 7 - DNA, 20 bp, Artificial Sequence
  Primer
tttttgcagagcaaggtgat

SEQ ID NO: 8 - DNA, 22 bp, Artificial Sequence
  Primer
aagcagtgcctggaaagtataa

SEQ ID NO: 9 - DNA, 20 bp, Artificial Sequence
  Primer
tggtctggtttcctctttgg

SEQ ID NO: 10 - DNA, 20 bp, Artificial Sequence
  Primer
gaagcaatccaaggtcacgt

SEQ ID NO: 11 - DNA, 20 bp, Artificial Sequence
  Primer
catgagctttgggggacata

SEQ ID NO: 12 - DNA, 19 bp, Artificial Sequence
  Primer
ccccacaccatggatttct

SEQ ID NO: 13 - DNA, 20 bp, Artificial Sequence
  Primer
gaatcacccaagaggctgac

SEQ ID NO: 14 - DNA, 20 bp, Artificial Sequence
  Primer
gaaggtcctttcccaaatgc

SEQ ID NO: 15 - DNA, 20 bp, Artificial Sequence
  Primer
tgtccgtttttgtcctccac

SEQ ID NO: 16 - DNA, 20 bp, Artificial Sequence
  Primer
gattacaggcgtgagccact

SEQ ID NO: 17 - DNA, 20 bp, Artificial Sequence
  Primer
cttgttggtcaggctggtct

SEQ ID NO: 18 - DNA, 30 bp, Artificial Sequence
  Primer
ctagtctagaaaggtgattcgcgaagagag

SEQ ID NO: 19 - DNA, 29 bp, Artificial Sequence
  Primer
tcccccgggactggtggtttttcctgtgc

SEQ ID NO: 20 - DNA, 21 bp, Artificial Sequence
  Primer
agtacactgcccttaccttgc